(19) United States
(12) Patent Application Publication
(10) Pub. No.: US 2013/0236450 A1
(43) Pub. Date: Sep. 12, 2013

(54) Methods Using Lipoprotein-Associated Phospholipase A2 in an Acute Care Setting

(71) Applicants: Joan Montaner, Barcelona (ES); Maria Pilar Delgado Martinez, Sant Joan Despi (ES)

(72) Inventors: Joan Montaner, Barcelona (ES); Maria Pilar Delgado Martinez, Sant Joan Despi (ES)

(21) Appl. No.: 13/695,027

(22) PCT Filed: May 2, 2011
(86) PCT No.: PCT/US11/34728

§ 371 Date: May 23, 2013

Related U.S. Application Data
(60) Provisional application No. 61/330,193, filed on Apr. 30, 2010.

(51) Int. Cl. C12Q 1/44; A61B 8/08; A61B 5/02

(52) U.S. Cl. CPC C12Q 1/44; A61B 5/02014; A61B 8/488; A61B 8/0891
USPC 424/133.1; 435/19; 600/481; 600/453

(57) ABSTRACT

This invention relates to methods for using Lipoprotein-associated Phospholipase A2 (Lp-PLA2) to care for subjects in an acute care setting. Specifically, Lp-PLA2 can be used determine if a subject having a vascular event, such as a stroke or heart attack, will benefit from therapy in the acute care setting. Moreover, it relates to methods of assessing risk and severity of a stroke by evaluating Lp-PLA2 levels alone or in combination with other assessments. In addition the invention relates to methods of using Lp-PLA2 to assess the functional outcome in a subject having a vascular event such as a stroke or heart attack.

This patent application claims the benefit of priority from U.S. Provisional Application Ser. No. 61/330,193, filed Apr. 30, 2010, which is hereby incorporated by reference in its entirety.

### FIELD OF THE INVENTION

This invention relates to methods for using Lipoprotein-associated Phospholipase A2 (Lp-PLA2) to care for subjects in an acute care setting. Specifically, Lp-PLA2 can be used determine if a subject having a vascular event, such as a stroke or heart attack, will benefit from therapy in the acute care setting. Moreover, it relates to methods of assessing risk and severity of a stroke by evaluating Lp-PLA2 levels alone or in combination with other assessments. In addition the invention relates to methods of using Lp-PLA2 to assess the functional outcome in a subject having a vascular event such as a stroke or heart attack.

### BACKGROUND OF THE INVENTION

### Introduction

Lipoprotein-associated Phospholipase A2 (Lp-PLA2) is an enzymatically active 50 kD protein that has been associated with Coronary vascular disease (CVD) including coronary heart disease (CHD) and stroke. Lp-PLA2 has been previously identified and characterized in the literature by Tew et al. (1996) Arterioscler. Thromb. Vasc. Biol. 16:591-599, Tjoelker, et al. (1995) Nature 374(6522):549-53), and Caslake et al. (2000) Atherosclerosis 150(2): 413-9. In addition, the protein, assays and methods of use have been described in the patent literature WO 95/00649-A1: U.S. Pat. Nos. 5,981,252, 5,968,818, 6,177,257, 7,052,862, 7,045,329, 7,217,535, 7,416,853; WO 00/24910-A1: U.S. Pat. Nos. 5,532,152; 5,605,801; 5,641,669; 5,656,431; 5,698,403; 5,977,308; and 5,847,088; WO 04/089184; WO 05/001416: U.S. Pat. No. 7,531,316; WO 05/074604; WO 05/113797; the contents of which are hereby incorporated by reference in their entirety. Lp-PLA2 is expressed by macrophages, with increased expression in atherosclerotic lesions (Hakkinin (1999) Arterioscler Thromb Vasc Biol 19(12): 2909-17). Lp-PLA2 circulates in the blood bound mainly to LDL, co-purifies with LDL, and is responsible for >95% of the phospholipase activity associated with LDL (Caslake 2000).

The United States Food and Drug Administration (FDA) has granted clearance for the PLAC® Test (diaDexus, South San Francisco, Calif.) for the quantitative determination of Lp-PLA2 in human plasma or serum, to be used in conjunction with clinical evaluation and patient risk assessment as an aid in predicting risk for coronary heart disease, and ischemic stroke associated with atherosclerosis.

Various methods for detecting Lp-PLA2 protein have been reported which include immunoassays (Caslake, 2000)., activity assays (PAF Acetylhydrolase Assay Kit, Cat#760901 product brochure, Cayman Chemical, Ann Arbor, Mich., Dec. 18, 1997 (caymanchem with the extension .com of the world wide web); Azwell/Alfresa Auto PAF-AH kit available from the Nesco Company, Alfresa, 2-24-3 Sho, Ibaraki, Osaka, Japan or Karlan Chemicals, Cottonwood, Ariz., see also Kosaka (2000)), spectrophotometric assays for serum platelet activating factor acetylhydrolase activity (Clin Chem Acta 296: 151-161, WO 00/32808 (to Azwell)). Other published methods to detect Lp-PLA2 include WO 00/032808, WO 03/048172, WO 2005/001416, WO 05/074604, WO 05/113797. The contents of the published applications are hereby incorporated by reference in their entirety.

### Stroke

Stroke is a leading cause of death and disability in the world. Worldwide there are 16 million first time strokes annually and 5.7 million stroke deaths. Eighty-seven percent of these deaths occur in low- and middle-income countries. Globally, there are more than 50 million survivors of stroke and transient ischemic attack (TIA). Of these survivors, at least 1 in 5 will have another stroke within 5 years (Strong K (2007) Lancet Neurol. 6:182-187).

In the United States stroke is the third-leading cause of death with about 150,000 per year. Only heart disease and cancer kill more people.

There are approximately 780,000 strokes per year, of which 600,000 are strokes occurring in patients for the first time and 180,000 are recurrent strokes. These attacks leave a large number of survivors with disabilities. Of the approximately 5-6 million stroke survivors in the United States 15%-30% of stroke victims experience permanent disability and 20% require institutional care at 3 months after onset. The total annual cost of stroke was estimated to be $62.7 billion in 2004 in the United States. See Heron (2007) National Vital Statistics Reports. 56(5):1-96 and Rosamond (2008) Circulation. 117:e25-e146. Accordingly, there is a great need to assess an individual's risk for stroke and to provide appropriate care for those who have had a stroke.

Data presented from the Rotterdam Study—Oei et al (European Society of Cardiology in August 2004) and from the ARIC Study—Ballantyne et al. (Scientific Sessions of the American Heart Association (AHA) in November 2004) indicate Lp-PLA2 is an independent risk factor for stroke. In addition, the ARM stroke study indicated that the measurement of both hsCRP and Lp-PLA2 was particularly useful for stroke risk assessment. After adjusting for traditional cardiovascular risk factors, lipids and hsCRP, elevated levels of Lp-PLA2 were associated with a doubling of risk for ischemic stroke. As in other stroke epidemiological studies, LDL cholesterol (LDL-C) did not differentiate stroke cases from controls in ARIC. Interestingly, statins lower risk of ischemic stroke (and levels of Lp-PLA2), even though LDL-C is not a reliable predictor of stroke (Ballantyne (2005) Arch Intern Med. 165:2479-2484).

Several studies have evaluated Lp-PLA2 and stroke in acute settings. Elkind et al (Arch Intern Med. 2006; 166:2073-2080) evaluated 467 patients with first-ever ischemic stroke who were followed for four years to determine whether levels of hs-CRP and Lp-PLA2 drawn in the setting of acute stroke (84% drawn within 72 hours of stroke) predict risk of stroke recurrence. Levels of Lp-PLA2 and hs-CRP were weakly correlated. After multivariate analysis, patients with the highest Lp-PLA2 levels had double the risk for recurrent stroke and for the combined outcome of stroke, MI, or vascular death. Lp-PLA2 identifies stroke patients who require the most aggressive treatment to prevent a second event. Cucchiara et al (Stroke. 2009 July; 40(7):2332-6) conclude that many patients with TIA have a high-risk mechanism (large vessel stenosis or cardioembolism) or will experience stroke/death within 90 days. The results from their study suggest a potential role for measuring Lp-PLA2 for short-term risk stratification of patients with acute TIA. A review article by Philip Gorelick (Am J Cardiol. 2008; 101[suppl]:34F-40F) provides the first published review of several important prospective epidemiological studies of Lp-PLA2 and risk of stroke. He finds that the “Lp-PLA2 immunoassay may prove to be especially useful for proper risk classification of persons with stroke or cardiovascular diseases who are found to be at moderate risk. It appears useful in overall cardiovascular risk classification and may lead to more aggressive therapeutic approaches with statin agents for lipid control or with other high-risk patient approaches for cardiovascular disease reduction.” Dr. Gorlick characterizes the findings of Furie et al. (Stroke 2007; 38:458) in a study evaluating Lp-PLA2 in patients with acute ischemic stroke stating “Lp-PLA2 was a significant predictor of risk of early stroke recurrence at 6 month and remained significant after multivariate adjustment for diabetes, hypertension, hyperlipidemia, atrial fibrillation, smoking and stroke subtype.”

While Lp-PLA2 has previously been shown to be associated with primary and secondary stroke and useful as a marker to assess risk of stroke, no data have shown Lp-PLA2 as a useful marker to select patients who will benefit from therapy in an acute setting.

### Coronary Heart Disease

Lipoprotein-associated phospholipase A2 (Lp-PLA2) levels have been shown to be significantly correlated in men with angiographically-proven Coronary Heart Disease (CHD) (Caslake 2000) and associated with cardiac events in men with hypercholesterolemia (Packard (2000) N Engl J Med 343(16): 1148-55).

Coronary heart disease (CHD) is the single most prevalent fatal disease in the United States. In the year 2003, an estimated 1.1 million Americans are predicted to have a new or recurrent coronary attack (see the American Heart Association web site, americanheart with the extension .org of the world wide web). Approximately 60% of these individuals have no previously known risk factors. It is apparent that there is a great need to diagnose individuals at risk of developing CHD, selecting patients suitable for therapy and monitoring response to therapies directed at reducing the individual's risk.

Coronary vascular disease (CVD) encompasses all diseases of the vasculature, including high blood pressure, coronary heart disease (CHD), stroke, congenital cardiovascular defects and congestive heart failure. Studies have shown that CHD is responsible for the majority of the CVD. The prevalence of CHD increases markedly as a function of age, with men having a higher prevalence than women within most age groups.

The current standard of care used to identify individuals at risk for heart disease is the measurement of a lipid panel, including triglycerides, total cholesterol, low density lipoprotein (LDL)-cholesterol, and high density lipoprotein (HDL)-cholesterol (Adult Treatment Panel III). Executive Summary of The Third Report of The National Cholesterol Education Program (NCEP) Expert Panel on Detection, Evaluation, And Treatment of High Blood Cholesterol In Adults (Adult Treatment Panel III). JAMA (2001) 285(19): 2486-97. According to the recent Adult Treatment Panel III (ATP III) guidelines (2001), depending on the risk factor score, individuals with LDL-cholesterol levels from ≧100 to ≦130 mg/dL are recommended to initiate therapeutic lifestyle changes. Adults with LDL-cholesterol >130 mg/dL are recommended for intensive lifestyle therapy and an LDL-cholesterol-lowering drug therapy to achieve an LDL-cholesterol goal of <100 mg/dL. Patients with LDL levels >160 mg/dL should be considered for therapies with lipid-lowering drugs. The American Heart Association has estimated that over 100 million adults in the US exceed the optimal level of total cholesterol. See the website americanheart with the extension .org of the world wide web.

While research continues to link elevated LDL-cholesterol levels with CHD risk, it is well understood that a significant number of individuals with normal LDL-cholesterol levels experience a cardiac event, suggesting that other factors not currently recognized may be involved (Eaton (1998) J Am Board Fam Pract 11(3): 180-6). In the search for new risk factors, significant attention has been focused in recent years on markers of inflammation, as a growing body of basic and clinical research emerges regarding the role of inflammation in atherogenesis (Lusis (2000) Atherosclerosis. Nature 407(6801): 233-41; Lindahl (2000) N Engl J Med 343(16): 1139-47). Some of the inflammatory markers under investigation include cell adhesion molecules, CD-40 ligand, interleukin 6 and C-reactive protein (CRP, measured by the high sensitivity method, or hsCRP). CRP, a non-specific acute phase inflammatory marker, has recently received significant attention as a potential risk indicator for CHD (Ridker (2002) N Engl J Med 347(20): 1557-65; Blake (2002)); J Intern Med 252(4): 283-94). CRP, however, is well known to be responsive to many sources of inflammation, which justifies further investigations to identify more specific markers of arterial involvement.

The pathogenesis of atherosclerosis leading to the formation of unstable plaque has been recognized as one of the major causes of CHD (Lusis 2000). Recently, new understanding of the pathogenesis of atherosclerosis has placed emphasis on the inflammatory process as a key contributor to the formation of unstable plaque. The instability of the atherosclerotic plaque, rather than the degree of stenosis, is considered to be the primary culprit in the majority of myocardial infarctions (MI). This realization has led to the investigation of plaque biology and recognition that markers of inflammation may be useful as predictors of cardiovascular risk. Among the various candidate markers of inflammation, CRP (measured by high sensitivity method, hs-CRP), a non-specific acute phase inflammatory marker, has received the most attention as a predictor of CHD (Ridker 2002).

### Peripheral Vascular Disease and Additional Diseases

Peripheral vascular disease (PVD) is a nearly pandemic condition that has the potential to cause loss of limb, or even loss of life. PVD manifests as insufficient tissue perfusion caused by existing atherosclerosis that may be acutely compounded by either emboli or thrombi. Because of the connection between Lp-PLA2, atherosclerosis and vascular inflammation, measurement of Lp-PLA2 levels may be useful for detecting, diagnosing or monitoring PVD. Recently, Santos et al. reported studies of Lp-PLA2 and ankle-brachial index (ABI) a measure of peripheral vascular disease. They found Lp-PLA2 was a borderline-significant predictor of lower ABI (p=0.05) whereas the other markers studied, CRP and white blood count (WBC), were not significant (Santos (2004) Vasc Med. 9(3):171-6).

Lp-PLA2 has been implicated in several other diseases including respiratory distress syndrome (Grissom (2003) Crit Care Med. 31(3):770-5), immunoglobulin A nephropathy (Yoon (2002) Clin Genet. 62(2):128-34), graft patency of femoropopliteal bypass (Unno (2002) Surgery 132(1):66-71), oral-inflammation (McManus and Pinckard (2000) Crit Rev Oral Biol Med. II (2):240-5 8), airway inflammation and hyperreactivity (Henderson (2000) J. Immunol. 15; 1 64(6):3360-7), HIV and AIDS (Khovidhunkit (1999) Metabolism 48(12):1524-31), asthma (Satoh (1999) Am J Respir Crit Care Med. 159(3):974-9), juvenile rheumatoid arthritis (Tselepis (1999) Arthritis Rheum. 42(2):373-83), human middle ear effusions (Tsuji (1998) ORL J Otorhinolaryngol Relat Spec. 60(1):25-9), schizophrenia (Bell (1997) Biochem Biophys Res Commun. 29; 241(3):630-5 9), necrotizing enterocolitis development (Muguruma, (1997) Adv Exp Med Biol. 407:3 79-82), and ischemic bowel necrosis (Furukawa (1993) Pediatr Res. 34(2):237-41).

### The Molecular Basis for Disease

Oxidation of LDL in the endothelial space of the artery is considered a critical step in the development of atherosclerosis. Oxidized LDL, unlike native LDL, has been shown to be associated with a host of pro-inflammatory and pro-atherogenic activities, which can ultimately lead to atherosclerotic plaque formation (Glass (2001) Cell 104(4): 503-16; Witztum (1994) Lancet 344(8925): 793-5). Increasing evidence from basic research suggests that atherosclerosis has an inflammatory component and represents much more than simple accumulation of lipids in the vessel wall. The earliest manifestation of a lesion is the fatty streak, largely composed of lipid-laden macrophages known as foam cells. The precursors of these cells are circulating monocytes. The ensuing inflammatory response can further stimulate migration and proliferation of smooth muscle cells and monocytes to the site of injury, to form an intermediate lesion. As layers of macrophages and smooth muscle cells accumulate, a fibrous plaque is formed, which is characterized by a necrotic core composed of cellular debris, lipids, cholesterol, calcium salts and a fibrous cap of smooth muscle, collagen and proteoglycans. Gradual growth of this advanced lesion may eventually project into the arterial lumen, impeding the flow of blood. Further progression of atherosclerosis may lead to plaque rupture and subsequent thrombus formation, resulting in acute coronary syndromes such as unstable angina, MI or sudden ischemic death (Davies (2000) Heart 83:361-366; Libby (1996) Curr Opin Lipidol 7(5): 330-5).

Lp-PLA2 plays a key role in the process of atherogenesis by hydrolyzing the sn-2 fatty acid of oxidatively modified LDL, resulting in the formation of lysophosphatidylcholine and oxidized free fatty acids (Macphee (1999) Biochem J 338 (Pt 2): 479-87). Both of these oxidized phospholipid products of Lp-PLA2 action are thought to contribute to the development and progression of atherosclerosis, by their ability to attract monocytes and contribute to foam cell formation, among other pro-inflammatory actions (Macphee (2001) Cuff Opin Pharmacol 1(2): 121-5; Macphee (2002) Expert Opin Ther Targets 6(3): 309-14).

### Clinical Studies

Lp-PLA2 has been previously reported as a potential risk factor for CHD. The predictive value of plasma levels of Lp-PLA2 for CHD has been reported in a large, prospective case-control clinical trial involving 6,595 men with hypercholesterolemia, known as the West of Scotland Coronary Prevention Study (WOSCOPS) (Packard 2000). Lp-PLA2 was measured in 580 CHD cases (defined by non-fatal MI, death from CHD, or a revascularization procedure) and 1,160 matched controls. The results indicated that plasma levels of Lp-PLA2 were significantly associated with development of CHD events by univariate and multivariate analyses, with almost a doubling of the relative risk for CHD events for the highest quintile of Lp-PLA2 compared to the lowest quintile. The association of Lp-PLA2 with CHD was independent of traditional risk factors such as LDL-cholesterol and other variables. This study provided an encouraging preliminary indication of the clinical utility of Lp-PLA2 as a risk factor for CHD.

Furthermore, in a study of angiographically proven CHD, Lp-PLA2 was shown to be significantly associated with the extent of coronary stenosis (Caslake 2000).

In another study, in which only females were examined (n=246, 123 cases and 123 controls), baseline levels of Lp-PLA2 were higher among cases than controls (p=0.016), but was not significantly associated with CHD when adjusted for other cardiovascular risk factors. In this study, cases included 40% of women with stroke, 51% non-fatal myocardial infarction and 9% fatal CHD (Blake (2001) J Am Coll Cardiol 38(5): 1302-6).

Recently, several large studies have added to the clinical evidence. For example, the Atherosclerosis Risk in Communities Study (ARIC) was designed to study, over a ten year period, the etiology, risk factors, clinical sequelae, and treatment alternatives for atherosclerosis. It was sponsored by the National Institutes of Health (NIH) and involved 15,792 apparently healthy men and women, aged 45 to 64, in four communities in the United States. In a retrospective study using banked samples, individuals with LDL <130 mg/dL but elevated levels of Lp-PLA2 (highest tertile) had a 2.08-fold higher risk of a coronary event compared to those individuals with low levels of Lp-PLA2 (Ballantyne (2004) Circulation. 109(7): 837-42).

Monitoring Trends and Determinants in Cardiovascular Diseases Study (MONICA) was a recent World Health Organization project collecting data from 282,279 apparently healthy men from urban and rural areas in twenty-one countries. In a subsequent study using serum samples from a sub-population of the MONICA subjects, the association between Lp-PLA2 and coronary events was investigated. In this sub-study, 934 men, aged 45 to 64, were followed for 14 years. Mean baseline levels of Lp-PLA2 were significantly higher in the cases versus the non-cases (p=0.01). A one standard deviation increase in Lp-PLA2 concentration as measured by an ELISA was associated in a univariate analysis with a relative risk of 1.37 (p=0.0002), and the risk association remained statistically significant even after adjusting for other factors such as age, diabetes, smoking, blood pressure, lipid levels, BMI and CRP level (relative risk: 1.21; p<0.04). In this study, individuals with the highest levels of both Lp-PLA2 and CRP had a 1.9-fold greater risk than individuals with low levels of both markers.

Lp-PLA2 has been cleared by the FDA for predicting risk for coronary heart disease, and ischemic stroke associated with atherosclerosis and these data support the utility of Lp-PLA2 to predict a first ever stroke and is beginning to be suggested as a marker to predict a second stroke or vascular event after a first cerebrovascular event.

Alberts et al showed at the ISC in New Orleans (Stroke. 2008; 39(2):642) a meta-analysis reviewing five published prospective epidemiological studies confirming the association of elevated Lp-PLA2 and the risk of stroke (Atherosclerosis Risk in Communities (ARIC), 2005, Healthy middle-aged adults; Rotterdam Study, 2005, Healthy men and women; Veterans Affairs HDL Intervention Trial (VA-HIT), 2006, Recurrent CV events, low LDL and low HDL; Women's Health Initiative Observational Study, 2008, Postmenopausal women; Malmö Diet and Cancer Study, 2008, 5393 (60% women) healthy subjects).

In a study evaluating recurrent strokes (Elkind et al, 2006) Lp-PLA2 was related with an increased risk of recurrent stroke (adjusted hazard ratio, 2.08; 95% confidence interval, 1.04-4.18) and of the combined outcome of recurrent stroke, MI, or vascular death (adjusted hazard ratio, 1.86; 95% confidence interval, 1.01-3.42). However in the study by Furie presented at the ISC 2007, an association was found for a recurrent stroke within the next 6 months after a first stroke 1.014 (1.3-6.6), but not for the combined endpoint of stroke, MI or vascular death.

### Care in the Acute Setting

The American Heart Association and American Stroke Association strongly urge people to seek medical attention as soon as possible if they believe they're having a stroke or heart attack. The sooner thrombolytic agents or other appropriate treatment is begun, the better the chances for recovery. One such thrombolytic agent is tissue plasminogen activator (tPA), a clot-busting drug. tPA is approved for use in certain patients having a heart attack or stroke. The drug can dissolve blood clots, which cause most heart attacks and strokes. tPA is the only drug approved by the U.S. Food and Drug Administration for the acute (urgent) treatment of ischemic stroke.

According to the American Heart Association studies have shown that thrombolytic agents, such as tPA, can reduce the amount of damage to the heart muscle and save lives. However, to be effective, they must be given within a few hours after symptoms begin. Administering tPA or other clot-dissolving agents is complex and is done through an intravenous (IV) line in the arm by hospital personnel. tPA has also been shown to be effective in treating ischemic stroke. This kind of stroke is caused by blood clots that block blood flow to the brain.

In 1996 the U.S. Food and Drug Administration (FDA) approved the use of tPA to treat ischemic stroke in the first three hours after the start of symptoms. This makes it very important for people who think they're having a stroke to seek help immediately. If given promptly, tPA can significantly reduce the effects of stroke and reduce permanent disability. tPA can only be given to a person within the first few hours after the start of stroke symptoms. The National Institute of Neurological Disorders and Stroke (NINDS) study suggested that 8 out of 18 stroke patients who receive tPA according to a strict protocol will recover by three months after the event without significant disability. This is compared to 6 out of 18 stroke patients (one-third) who recover substantially regardless of treatment. (N Engl J Med 333:1581-1587, 1995.)

While tPA or other thrombolytics can reduce disability from a heart attack or stroke, there is also a higher risk of bleeding. Studies vary in predicting the likelihood of complications, which include bleeding into the brain, other types of serious bleeding (e.g., gastrointestinal), and death. The NINDS study suggested that bleeding into the brain occurred in about 1 out of 18 patients receiving tPA (specifically, 5.8%). When this occurred, there was a 45 percent fatality rate. Several studies suggested treatment with “clot-dissolving” medications increases the number of patients who die following a stroke (JAMA 274(13):1017, 1995; Lancet 346:1509-1514, 1995; JAMA 276(12):961-6, 1996; NEJM 335(3):145, 1996; Lancet 352:1245-1251, 1998; JAMA 282(21):2019-26. 1999). Subsequent studies demonstrated that using tPA more liberally than is recommended in the NINDS protocol resulted in a higher rate of intracranial hemorrhage (JAMA 283:1151-1158, 2000; Cerebrovasc Dis 8(suppl 4):48, 1998; Arch Intern Med 162:1994-2001, 2002; Cochrane Database Syst Rev. 2000:CD000213; Cochrane Database Syst Rev. 2000:CD000029). Complications are more likely when tPA is used in patients over 70 years old, those with more severe stroke, or those with glucose over 300 mg/dl.

Due to the severe risks associated with thrombolytics, it is important for physicians to weigh the possibility of benefit (e.g. improved function at 3 months) against the possibility of harm (severe bleeding or death). Stroke symptoms alone are insufficient to definitely diagnose stroke and, in patients with a stroke mimic, tPA use results only in potential adverse effects without any possibility of benefit. It is clear there is a need to identify patients who are suspected of having a cardiovascular event who will benefit from administration of thrombolytics (e.g. tPA).

Lp-PLA2 Inhibitors

Several papers have been published citing the potential of Lp-PLA2 as a therapeutic target for the treatment of coronary artery disease and atherosclerosis (Caslake 2000; Macphee 2001; Carpenter (2001) FEBS Lett. 505(3):357-63.; Leach (2001) Farmaco 56(1-2): 45-50). Evidence that Lp-PLA2 is a therapeutic target for the treatment of CHD has been published in many articles describing several genuses of inhibitors of Lp-PLA2 and their use. These genuses include but are not limited to: azetidinone inhibitors, SB-222657, SB-223777 (MacPhee 1999); reversible 2-(alkylthio)-pyrimidin-4-ones (Boyd et al. (2000) Bioorg Med Chem Lett. 10(4):395-8); natural product derived inhibitors, SB-253514 and analogues (Pinto (2000); Bioorg Med Chem Lett. 10(17):2015-7); inhibitors produced by Pseudomonas fluorescens DSM 11579, SB-253514 and analogues (Thirkettle (2000) et al. J Antibiot (Tokyo). 53(7):664-9; Busby (2000) J Antibiot (Tokyo). 53(7):670-6.; Thirkettle (2000) J Antibiot (Tokyo). 53(7):733-5); 2-(alkylthio)-pyrimidones, orally active 1-((amidolinked)-alkyl)-pyrimidones (Boyd et al. (2000) Bioorg Med Chem Lett. 10(22):2557-61); modified pyrimidone 5-substituent in 1-((amidolinked)-alkyl)-pyrimidones is highly water soluble (Boyd, et al. (2001) Bioorg Med Chem Lett. 2001 11(5):701-4); phenylpiperazineacetamide derivative of lipophilic 1-substituent in 1-((amidolinked)-alkyl)-pyrimidones (Bloomer (2001) Bioorg Med Chem Lett. 11(14):1925-9.); 5-(Pyrazolylmethyl) derivative and 5-(methoxypyrimidinylmethyl) derivative of 1-(biphenylmethylamidoalkyl)-pyrimidones (Boyd et al. (2002) Bioorg Med Chem Lett. 12(1):51-5); cyclopentyl fused derivative, SB-480848, of the pyrimidone 5-substituent in clinical candidate SB-435495 (Blackie (2003) Bioorg Med Chem Lett. 2003 Mar. 24; 13(6):1067-70). To date, GlaxoSmithKline (GSK) has announced positive clinical data for a novel compound, darapladib, that dramatically lowers Lp-PLA2 activity. Darapladib and other Lp-PLA2 inhibitors, including ralapladib, may represent a new generation of drugs that reduce cardiovascular disease and death.

### Lp-PLA2 and Other Therapeutic Molecules

Winkler recently reported a multicenter, double-blind, randomized study evaluating the effects of fluvastatin XL versus placebo on the level of Lp-PLA2 in 89 patients with type 2 diabetes (42 fluvastatin and 47 placebo) (Winkler (2004) J Clin Endocrinol Metab. 89(3) 1153-1159). Among these subjects, higher Lp-PLA2 activity was significantly associated with a history of CAD. The highest quartile in terms of Lp-PLA2 activity was at significantly greater risk than the lowest quartile (risk ratio: 2.09; 95% CI: 1.02-4.29; p=0.043). Fluvastatin treatment decreased Lp-PLA2 activity by 22.8%. Blankenberg also reported that taking statins lowered the measurable Lp-PLA2 activity (Blankenberg (2003) J of Lipid Research 44: 1381-1386).

Albert et al reported on the effect of statin therapy on lipoprotein associated phospholipase a2 levels. The researchers evaluated the effect of pravastatin 40 mg daily vs. placebo on Lp-PLA2 levels in a cardiovascular disease free population derived from the PRINCE trial. After 12 weeks, Lp-PLA2 levels decreased by 22.1% among treated patients (vs. 7.8% among placebo group). Only 6% of the lowering of Lp-PLA2 by pravastatin could be accounted for by the lowering of LDL-C (Albert (2005) Atherosclerosis. 182:193-198).

Schaefer et al reported on the effects of atorvastatin versus other statins on fasting and postprandial c-reactive protein and Lp-PLA2 in patients with coronary heart disease versus control subjects. In this study the impact of various statins at the 40 mg/day dosage on Lp-PLA2 was compared. The study found that “atorvastatin is more effective than fluvastatin, lovastatin, pravastatin, or simvastatin for decreasing not only low density lipoprotein cholesterol but also hs-CRP and Lp-PLA2” (Schaefer (2005) Am J Cardiol. 95:1025-1032).

Saougos et al have reported on the effect of hypolipidemic drugs on Lp-PLA2. This is the first study to demonstrate that ezetimibe and rosuvastatin both lower Lp-PLA2 mass. Statin intolerant Type IIa dyslipidemics had an 18% reduction in Lp-PLA2 mass with ezetimibe 10 mg/day, and Type IIa dyslipidemics had a 29% reduction in Lp-PLA2 mass with rosuvastatin 10 mg/day. It also showed that fenofibrate 200 mg/day lowered Lp-PLA2 mass 32%, a finding similar to fenofibrate's effect on Lp-PLA2 mass in Type 2 DM (Saogos (2007) Arterioscler Thromb Vase Biol. 27:2236-2243).

Muhlestein et al reported on The Reduction of Lp-PLA2 by statin, fibrate, and combination therapy among diabetic patients with mixed dyslipidemia. This study evaluated the effect of simvastatin 20 mg and fenofibrate 160 mg on Lp-PLA2 and CRP in type 2 diabetic patients with mixed dyslipidemia. Fenofibrate, simvastatin and the combination each lowered Lp-PLA2, and the effect was greatest among patients with baseline levels greater than the median. In this study, lipid-modifying agents lowered Lp-PLA2 by more than 25% (fenofibrate: 27%; simvastatin: 35%) (Muhlestein (2006) J Am Coll Cardiol. 48:396-401).

Rosenson et al recently reported on the effects of fenofibrate on Lp-PLA2 levels in non-diabetic patients with metabolic syndrome. In this study reduction in small LDL-P particles was significantly associated with the reduction in Lp-PLA2, suggesting that fenofibrate may lower Lp-PLA2 via plaque stabilization mediated by lowering small LDL-P (Rosenson (2008) Am Heart J. 155(3):499.e9-16.).

Schmidt et al reported on the effects of eicosapentaenoic acid (EPA) on Lp-PLA2 levels in patients admitted to elective coronary angiography because of suspected coronary artery disease (CAD). The content of the marine n-3 fatty acid, eicosapentaenoic acid (EPA) in adipose tissue, a measure of long-term intake of seafood independently and inversely correlated with plasma levels of Lp-PLA2 (r=−0.18, p<0.01). The results support that Lp-PLA2 may relate to CAD and that intake of marine n-3 fatty acids might reduce plasma Lp-PLA2 suggesting another mechanism by which n-3 fatty acids could reduce the risk of cardiovascular disease.

Kuvin et al reported on effects of extended-release niacin on lipoprotein particle size, distribution, and inflammatory markers in patients with coronary artery disease. This study evaluated the effect on Lp-PLA2 of adding niacin to stable coronary heart disease patients with well-managed baseline LDL levels of 76 mg/dL. While there was no significant change in baseline LDL levels after three months, niacin significantly lowered Lp-PLA2 by 20% (Kuvin (2006) Am J Cardiol. 98:743-745).

It appears from this study that Lp-PLA2 lowering was independent of LDL (which did not change) and that there appears to be residual opportunity to lower Lp-PLA2 in patients with low achieved LDL cholesterol, consistent with the concept that low achieved LDL alone may not assure that plaque has stabilized.

These studies identify therapies which benefit patients who have an increased risk of CVD including coronary heart disease and stroke.

All publications and other materials described herein are used to illuminate the invention or provide additional details respecting the practice and are hereby incorporated by reference in their entirety.

### SUMMARY OF THE INVENTION

An aspect of the present invention relates to a method for selecting a thrombolytic therapy for a subject comprising the steps of determining the level of Lipoprotein-associated Phospholipase A2 (Lp-PLA2) in the subject.

Another aspect of the present invention relates to a method for selecting a thrombolytic therapy for a subject, who has or is suspected of having coronary vascular disease (CVD), comprising the steps of determining the level of Lipoprotein-associated Phospholipase A2 (Lp-PLA2) in the subject.

Another aspect of the present invention relates to a method for selecting a thrombolytic therapy for a subject comprising the steps of determining the level of Lipoprotein-associated Phospholipase A2 (Lp-PLA2) in the subject and determining if the subject has a proximal vascular lesion or occlusion.

In these methods a low level of Lp-PLA2 indicates a subject likely to benefit from thrombolytic therapy while a high level of Lp-PLA2 indicates a subject likely to benefit from aggressive thrombolytic therapy, drug combinations and/or interventional and surgical approaches.

Another aspect of the present invention relates to a method of selecting a subject for therapeutic intervention comprising determining the level of Lipoprotein-associated Phospholipase A2 (Lp-PLA2) and the presence of a proximal vascular lesion or occlusion in the subject and selecting the subject with a high Lp-PLA2 level and a proximal vascular lesion or occlusion for therapeutic intervention.

Another aspect of the present invention relates to a method of selecting a subject, who has or is suspected of having coronary vascular disease (CVD), for therapeutic intervention comprising determining the level of Lipoprotein-associated Phospholipase A2 (Lp-PLA2) and the presence of a proximal vascular lesion or occlusion in the subject and selecting the subject with a high Lp-PLA2 level and a proximal vascular lesion or occlusion for therapeutic intervention.

Another aspect of the present invention relates to a method of assessing the functional outcome of a subject who has had or is suspected of having a myocardial infarction, stroke, TIA or cerebrovascular accident (CVA) comprising determining the level of Lp-PLA2 and the presence of a proximal vascular lesion or occlusion in the subject wherein the functional outcome of a subject with a high Lp-PLA2 level and a proximal vascular lesion or occlusion is functional dependence.

Yet another aspect of the present invention relates to a method of selecting a subject for therapeutic intervention comprising assessing a subject for functional outcome wherein a subject assessed to have functionally dependent outcome is selected for therapeutic intervention.

### BRIEF DESCRIPTION OF THE FIGURES

FIGS. 1A and 1B show the Lp-PLA2 mass and activity temporal profile in acute stroke.

FIGS. 2A and 2B show the Lp-PLA2 mass and activity temporal profile from baseline to the 3rd month.

FIGS. 3A and 3B show the Lp-PLA2 levels in healthy controls (n=135) and pooled samples from baseline, 1 hour and 24 hours (n=35 in each time) after baseline.

FIGS. 4A and 4B show the association of Lp-PLA2 mass and activity levels to admission NIHSS scores and stroke etiology.

FIGS. 4C and 4D show the association of Lp-PLA2 mass and activity levels to the location of occlusion and 1-hour recanalization.

FIGS. 4E and 4F show the association of Lp-PLA2 mass and activity levels to early neurological status and functional outcome at follow-up (third month).

FIG. 5 shows the relationship between location of vessel occlusion and Lp-PLA2 level and successful 1-hour complete recanalization.

FIG. 6 shows the relationship between location of vessel occlusion and Lp-PLA2 level and third month-functional outcome.

FIG. 7 shows levels of Lp-PLA2 mass and activity (boxplots) in TIA cases and controls.

FIGS. 8A and 8B show Kaplan-Meier curves demonstrating survival analyses for the presence of further vascular events or stroke/TIA considering ABCD2 score.

FIGS. 9A, 9B, 9C and 9D show Kaplan-Meier curves showing survival analyses for presence of further vascular events or stroke/TIA considering Lp-PLA2 activity (highest versus the lowest quartile of Lp-PLA2 activity).

FIGS. 9E, 9F, 9G and 9H show Kaplan-Meier curves showing survival analyses for presence of further vascular events or stroke/TIA considering Lp-PLA2 activity (cases above or below an optimal cut-off point of Lp-PLA2 activity).

FIGS. 10A and 10B show scatterplots of the correlation between Lp-PLA2 mass and activity and total cholesterol.

FIGS. 11A and 11B show scatterplots of the correlation between Lp-PLA2 mass and activity and LDL-cholesterol.

FIG. 12 shows Kaplan-Meier curves showing the cumulative survival of any vascular event during follow-up between 1th and a combination of 3rd and 4th quartiles of Lp-PLA2 activity.

FIG. 13 shows Kaplan-Meier curves show the cumulative survival of any vascular event during follow-up between groups above and under Lp-PLA2 activity cutpoint levels.

FIG. 14 shows the vascular risk stratification using the combination of Lp-PLA2 and ABI.

### DETAILED DESCRIPTION OF THE INVENTION

Lp-PLA2 can be used to identify patients who will benefit from administration of thrombolytics. Lp-PLA2 expression has been shown to be higher in carotid plaques of patients with than without cardiac events (Herrmann (2009) Eur Heart J. 30(23):2930-8). In the event of a plaque rupture and vascular thrombus, high levels of Lp-PLA2 may be released into circulation from the rupture site. Measuring Lp-PLA2 levels of individuals suspected of having a stroke or myocardial infarction (e.g. individuals who present symptoms of a stroke or MI) can identify individuals who will benefit from standard thrombolytic therapy or and those who may need aggressive therapy including aggressive thrombolytic drug dosing, drug combinations and/or interventional and surgical therapies.

This invention is directed to methods of using Lp-PLA2 levels to select patients for therapy, assess risk of cerebrovascular accident (CVA), and assess functional outcome for patients.

As used herein, the terms “embodiment” and “aspect” are used interchangeably.

As used herein, the term “coronary vascular disease” or “CVD” means diseases of the vasculature, including high blood pressure, coronary heart disease (CHD), myocardial infarction, stroke, transient ischemic attack (TIA), cerebrovascular accident (CVA), congenital cardiovascular defects and congestive heart failure. Coronary vascular disease includes primary and subsequent acute events including myocardial infarction, stroke, TIA and CVA.

“Lipoprotein-associated Phospholipase A2”, “Lp-PLA2”, “LpPLA2”, “Lp-PLA₂”, “Platelet-activating factor-acetylhydrolase”, “PAF-AH”, and “LDL-PLA2” are used interchangeably herein and within the literature and refer to native Lp-PLA2, and allelic variants thereof, as described, for example, in Tew et al. (1996) Arterioscler. Thromb. Vase. Biol. 16:591-599, Tjoelker, et al. (1995) Nature 374(6522):549-53), Caslake et al. (2000) Atherosclerosis 150(2): 413-9, Genbank RefSeq IDs: NM_{—}005084, NP_{—}005075, NM_{—}001168357 and NP_{—}001161829 and Genebank Entrez GenelD: 7941 (PLA2G7), which are hereby incorporated by reference in their entirety. Unless indicated otherwise, the terms “Lipoprotein-associated Phospholipase A2”, “Lp-PLA2”, “LpPLA2”, “Lp-PLA₂”, “Platelet-activating factor-acetylhydrolase”, “PAF-AH”, and “LDL-PLA2” when used herein refer to the human protein.

As used herein, the term “acute care” means health-care or necessary treatment of a disease over a short period of time in which a patient is treated for a brief but severe episode of illness, such as CVD, myocardial infarction and stroke. Acute care is typically rendered in an emergency department, ambulatory care clinic, or other short-term stay facility. An acute care setting or timeframe means within half an hour, 1 hour, 2 hours, 3 hours, 4 hours, 5 hours or 6 hours.

As used herein, the term “functional outcome” means the classification system that summarizes the neurological impairments, disabilities, and handicaps that occur after a vascular event such as stroke. The functional outcome for stroke encompasses a broad range of disabilities and impairments as well as the relationship of disability and impairment to independent function. Typically indicators for functional outcome are measured at 1 month, 3 months or 6 months after an acute vascular event, such as stroke. See Stroke. 1998; 29:1274-1280, which is hereby incorporated by reference in its entirety. Generally, a functional outcome of functional dependence is a poor outcome from a vascular event, such as stroke, in which the subject suffers from impairment, disability, handicap or compromised quality of life. The likelihood of a subject having a functional outcome of functional dependence after a vascular event, such as a stroke, can be reduced by aggressive therapy, including surgery, at the time of the vascular event in the acute care setting.

“High” refers to a measure that is greater than normal, greater than a standard such as a predetermined measure or a subgroup measure or that is relatively greater than another subgroup measure. For example, high Lp-PLA2 refers to a measure of Lp-PLA2 that is greater than a normal Lp-PLA2 measure. A normal Lp-PLA2 measure may be determined according to any method available to one skilled in the art. High Lp-PLA2 may also refer to a measure that is equal to or greater than a predetermined measure, such as a predetermined cutoff. High Lp-PLA2 may also refer to a measure of Lp-PLA2 wherein a high Lp-PLA2 subgroup has relatively greater levels of Lp-PLA2 than another subgroup. For example, without limitation, according to the present specification, two distinct patient subgroups can be created by dividing samples around a mathematically determined point, such as, without limitation, a median, thus creating a subgroup whose measure is high (ie, higher than the median) and another subgroup whose measure is low. Lp-PLA2 can be measured by any method known to one skilled in the art such as, for example, without limitation, using the PLAC® Test, an Lp-PLA2 activity assay, an immunohistochemical (IHC) assay or using any standard method for detecting Lp-PLA2, including Lp-PLA2 mass and Lp-PLA2 activity. In some cases, a “high” expression level may comprise a range of expression that is very high and a range of expression that is “moderately high” where moderately high is a level of expression that is greater than normal, but less than “very high”. Example ranges for high (including very high and moderately high) high Lp-PLA2 expression are provided in the literature cited herein, the PLAC Test product specification, in the present application and include >200 ng/mL, >201 ng/mL, >201.5 ng/mL, >210 ng/mL, >220 ng/mL, >230 ng/mL, >240 ng/mL, >250 ng/mL, >260 ng/mL, >270 ng/mL, >280 ng/mL, >290 ng/mL, >300 ng/mL, >100 ng/mL/min, >110 ng/mL/min, >120 ng/mL/min, >130 ng/mL/min, >140 ng/mL/min, >150 ng/mL/min, >160 ng/mL/min, >170 ng/mL/min, >180 ng/mL/min, >190 ng/mL/min, and >200 ng/mL/min.

“Likely to” (and “unlikely to”), as used herein, refers to an increased (or decreased) probability that an item, object, thing or person will occur. Thus, in one example, a subject that is likely to benefit from treatment with a thrombolytic agent has an increased probability of benefiting from treatment with a thrombolytic agent relative to a reference subject or group of subjects.

“Long,” as used herein, refers to a time measure that is greater than normal, greater than a standard such as a predetermined measure or a subgroup measure that is relatively longer than another subgroup measure. For example, with respect to a patient's longevity, a long time progression refers to time progression that is longer than a normal time progression. Whether a time progression is long or not may be determined according to any method available to one skilled in the art. In one embodiment, “long” refers to a time that is greater than the median time course required for a significant event to occur in a disease.

“Low” is a term that refers to a measure that is less than normal, less than a standard such as a predetermined measure or a subgroup measure that is relatively less than another subgroup measure. For example, low Lp-PLA2 means a measure of Lp-PLA2 that is less than a normal Lp-PLA2 measure in a particular set of samples of patients. A normal Lp-PLA2 measure may be determined according to any method available to one skilled in the art. Low Lp-PLA2 may also mean a measure that is less than a predetermined measure, such as a predetermined cutoff. Low Lp-PLA2 may also mean a measure wherein a low Lp-PLA2 subgroup is relatively lower than another subgroup. For example, without limitation, according to the present specification, two distinct patient subgroups can be created by dividing samples around a mathematically determined point, such as, without limitation, a median, thus creating a group whose measure is low (i.e., less than the median) with respect to another group whose measure is high (i.e., greater than the median). Lp-PLA2 can be measured by any method known to one skilled in the art such as, for example, without limitation, using the PLAC® Test, an Lp-PLA2 activity assay, an immunohistochemical (IHC) assay or using any standard method for detecting Lp-PLA2, including Lp-PLA2 mass and Lp-PLA2 activity. Example ranges for low values of Lp-PLA2 expression are provided in the literature cited herein, the PLAC Test product specification, in the present application and include <200 ng/mL, <201 ng/mL, <201.5 ng/mL, <210 ng/mL, <220 ng/mL, <230 ng/mL, <240 ng/mL, <250 ng/mL, <260 ng/mL, <270 ng/mL, <280 ng/mL, <290 ng/mL, <300 ng/mL, <100 ng/mL/min, <110 ng/mL/min, <120 ng/mL/min, <130 ng/mL/min, <140 ng/mL/min, <150 ng/mL/min, <160 ng/mL/min, <170 ng/mL/min, <180 ng/mL/min, <190 ng/mL/min, and <200 ng/mL/min.

“Overall survival” or “OS” refers to a time as measured from the start of treatment to death or censor. Censoring may come from a study end or change in treatment. Overall survival can refer to a probability as, for example, a probability when represented in a Kaplan-Meier plot of being alive at a particular time, that time being the time between the start of the treatment to death or censor.

“Pre-determined cutoff” as used herein, refers to the value of a predetermined measure on subjects exhibiting certain attributes that allow the best discrimination between two or more categories of an attribute. For example, a pre-determined cutoff that allows one to discriminate between two categories such as high Lp-PLA2 expression and low Lp-PLA2 expression for determining overall survival may be used. Pre-determined cutoffs may be used to separate the subjects with values lower than or higher than the pre-determined cutoff to optimize the prediction model.

“Respond” to treatment, and other forms of this verb, as used herein, refer to the reaction of a subject to treatment with an agent. As an example, a subject responds to treatment with an agent if the subject experiences a life expectancy extended by about 5%, 10%, 20%, 30%, 40%, 50% or more beyond the life expectancy predicted if no treatment is administered. In another example, a subject responds to treatment with an agent if the subject has an overall survival or increased time to progression. Several methods may be used to determine if a patient responds to a treatment.

“Sample” or “tissue sample” or “patient sample” or “patient cell or tissue sample” or “specimen” each refers to a collection of similar cells obtained from a tissue of a subject or patient. The source of the tissue sample may be solid tissue as from a fresh tissue, frozen and/or preserved organ or tissue or biopsy or aspirate; blood or any blood constituents, bodily fluids such as cerebral spinal fluid, amniotic fluid, peritoneal fluid or interstitial fluid or cells from any time in gestation or development of the subject. The tissue sample may contain compounds that are not naturally intermixed with the tissue in nature such as preservatives, anticoagulants, buffers, fixatives, nutrients, antibiotics or the like. Cells may be fixed in a conventional manner, such as in an FFPE manner.

“Short,” as used herein, refers to a time measure that is shorter than normal, shorter than a standard such as a predetermined measure or a subgroup measure that is relatively shorter than another subgroup measure. For example, with respect to a patient's longevity, a short time progression refers to time progression that is shorter than a normal time progression or shorter than predicted. Whether a time progression is short or not may be determined according to any method available to one skilled in the art. In one embodiment, “short” refers to a time that is less than the median time course required for a significant event to occur in a disease.

“Significant event,” as used herein, shall refer to an event in a patient's disease that is important as determined by one skilled in the art. Examples of significant events include, for example, without limitation, primary diagnosis, myocardial infarction, stroke, TIA, CVA, death, recurrence, the determination that a patient's disease is metastatic, relapse of a patient's disease or the progression of a patient's disease from any one of the above noted stages to another. A significant event may be any important event used to assess OS, TTP and/or other response criteria, as determined by one skilled in the art.

As used herein, the terms “subject” and “patient” are used interchangeably. As used herein, the terms “subject” and “subjects” refer to an animal, preferably a mammal including a non-primate (e.g., a cow, pig, horse, donkey, goat, camel, cat, dog, guinea pig, rat, mouse or sheep) and a primate (e.g., a monkey, such as a cynomolgus monkey, gorilla, chimpanzee or a human).

As used herein, “time course” shall refer to the amount of time between an initial event and a subsequent event. For example, with respect to a patient's cancer, time course may relate to a patient's disease and may be measured by gauging significant events in the course of the disease, wherein the first event may be diagnosis and the subsequent event may be a significant event, for example.

“Time to progression” or “TTP” refers to a time as measured from the start of the treatment to progression or a significant event or censor. Censoring may come from a study end or from a change in treatment. Time to progression can also be represented as a probability as, for example, in a Kaplan-Meier plot where time to progression may represent the probability of being progression free over a particular time, that time being the time between the start of the treatment to progression or censor.

“Treatment,” and other forms of this word, including “therapy”, refer to the administration of an agent to impede a disease, such as progression of CVD, to cause a reduction in risk for CVD, to extend the expected survival time of the subject and/or time to progression of the CVD or the like. Treatment may also refer to any course which one skilled, for example, a treating physician, deems expedient.

“Chemotherapeutic agent” means a chemical substance that is used to treat a condition, particularly cardiovascular disease.

As used herein, the term “metabolic disorder” includes a disorder, disease or condition which is caused or characterized by an abnormal metabolism (i.e., the chemical changes in living cells by which energy is provided for vital processes and activities) in a subject. Metabolic disorders include diseases, disorders, or conditions associated with hyperglycemia or aberrant adipose cell (e.g., brown or white adipose cell) phenotype or function. Metabolic disorders can detrimentally affect cellular functions such as cellular proliferation, growth, differentiation, or migration, cellular regulation of homeostasis, inter- or intra-cellular communication; tissue function, such as liver function, renal function, or adipocyte function; systemic responses in an organism, such as hormonal responses (e.g., insulin response). Examples of metabolic disorders include obesity, diabetes, hyperphagia, endocrine abnormalities, triglyceride storage disease, Bardet-Biedl syndrome, Lawrence-Moon syndrome, Prader-Labhart-Willi syndrome, anorexia, and cachexia. Obesity is defined as a body mass index (BMI) of 30 kg/m.sup.2 or more (National Institute of Health, Clinical Guidelines on the Identification, Evaluation, and Treatment of Overweight and Obesity in Adults (1998)). However, the invention is also intended to include a disease, disorder, or condition that is characterized by a body mass index (BMI) of 25 kg/m2 or more, 26 kg/m2 or more, 27 kg/m.sup.2 or more, 28 kg/m.sup.2 or more, 29 kg/m.sup.2 or more, 29.5 kg/m.sup.2 or more, or 29.9 kg/m.sup.2 or more, all of which are typically referred to as overweight (National Institute of Health, Clinical Guidelines on the Identification, Evaluation, and Treatment of Overweight and Obesity in Adults (1998)).

As used herein, “greater than or equal to” (i.e., ≧ or >=) can in certain alternative

embodiments mean “greater than” (>). Also, as used herein, “less than or equal to” (i.e., ≦ or <=) can in certain alternative embodiments mean “less than” (<).

Agents for reducing the risk of a Coronary Vascular Disorder include those selected from the group consisting of Lp-PLA2 inhibitors (Leach 2001), anti-inflammatory agents, anti-thrombotic agents, anti-platelet agents, fibrinolytic agents, lipid reducing agents, niacin, direct thrombin inhibitors, and glycoprotein II b/IIIa receptor inhibitors and agents that bind to cellular adhesion molecules and inhibit the ability of white blood cells to attach to such molecules (e.g. anti-cellular adhesion molecule antibodies).

Anti-inflammatory agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; C₁₋cloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium.

Anti-thrombotic and/or fibrinolytic agents include Plasminogen (to plasmin via interactions of prekallikrein, kininogens, Factors XII, XIIIa, plasminogen proactivator, and tissue plasminogen activator[TPA]) Streptokinase; Urokinase: Anisoylated Plasminogen-Streptokinase Activator Complex; Pro-Urokinase; (Pro-UK); rTPA (alteplase or activase; r denotes recombinant), rPro-UK; Abbokinase; Eminase; Sreptase Anagrelide Hydrochloride; Bivalirudin; Dalteparin Sodium; Danaparoid Sodium; Dazoxiben Hydrochloride; Efegatran Sulfate; Enoxaparin Sodium; Ifetroban; Ifetroban Sodium; Tinzaparin Sodium; retaplase; Trifenagrel; Warfarin; Dextrans.

Anti-platelet agents include Clopridogrel; Sulfinpyrazone; Aspirin; Dipyridamole; Clofibrate; Pyridinol Carbamate; PGE; Glucagon; Antiserotonin drugs; Caffeine; Theophyllin Pentoxifyllin; Ticlopidine; Anagrelide. Lipid reducing agents include gemfibrozil, cholystyramine, colestipol, nicotinic acid, probucol lovastatin, fluvastatin, simvastatin, atorvastatin, pravastatin, cirivastatin (for statins, see Crouch 2000). Direct thrombin inhibitors include hirudin, hirugen, hirulog, agatroban, PPACK, thrombin aptamers. Glycoprotein IIb/IIIa receptor Inhibitors are both antibodies and non-antibodies, and include but are not limited to ReoPro (abcixamab), lamifiban, tirofiban. One preferred agent is aspirin.

Markers of systemic inflammation, such as CRP, are well-known to those of ordinary skill in the art. It is preferred that the markers of systemic inflammation be selected from the group consisting of C-reactive protein, cytokines, and cellular adhesion molecules. Cytokines are well-known to those of ordinary skill in the art and include human interleukins 1-17. Cellular adhesion molecules are well-known to those of ordinary skill in the art and include integrins, ICAM-1, ICAM-3, BL-CAM, LFA-2, VCAM-1, NCAM, and PECAM. The preferred adhesion molecule is soluble intercellular adhesion molecule (sICAM-1).

The level of the markers of this invention may be obtained by a variety of recognized methods. Typically, the level is determined by measuring the level of the marker in a body fluid, for example, blood, lymph, saliva, urine and the like. The preferred body fluid is blood. The level can be determined by ELISA, or immunoassays or other conventional techniques for determining the presence of the marker. Conventional methods include sending samples of a patient's body fluid to a commercial laboratory for measurement. For the measurement of Lp-PLA2 enzymatic assays may also be used, see U.S. Pat. No. 5,981,252 or 5,880,273, the contents of which are hereby incorporated by reference into the subject application.

The invention also involves comparing the level of marker for the individual with a predetermined value. The predetermined value can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, e.g., tertiles, such as-a low-risk group, a medium-risk group and a high-risk group, or into quadrants, the lowest quadrant being individuals with the lowest risk and the highest quadrant being individuals with the highest risk.

In preferred embodiments the invention provides novel kits or assays which are specific for, and have appropriate sensitivity with respect to, predetermined values selected on the basis of the present invention. The preferred kits, therefore, would differ from those presently commercially available, by including, for example, different cut-offs, different sensitivities at particular cut-offs as well as instructions or other printed material for characterizing risk based upon the outcome of the assay.

As discussed above the invention provides methods for evaluating the likelihood that an individual will benefit from treatment with an agent for reducing risk of a future cardiovascular disorder. This method has important implications for patient treatment and also for clinical development of new therapeutics. Physicians select therapeutic regimens for patient treatment based upon the expected net benefit to the patient. The net benefit is derived from the risk to benefit ratio. The present invention permits selection of individuals who are more likely to benefit by intervention, thereby aiding the physician in selecting a therapeutic regimen. This might include using drugs with a higher risk profile where the likelihood of expected benefit has increased. Likewise, clinical investigators desire to select for clinical trials a population with a high likelihood of obtaining a net benefit. The present invention can help clinical investigators select such individuals. It is expected that clinical investigators now will use the present invention for determining entry criteria for clinical trials.

An effective amount is a dosage of the therapeutic agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health practitioner. For example, an effective amount can depend upon the degree to which an individual has abnormally elevated levels of markers of systemic information. It should be understood that the anti-inflammatory agents of the invention are used to prevent cardiovascular disorders, that is, they are used prophylactically in subjects at risk of developing a cardiovascular disorder. Thus, an effective amount is that amount which can lower the risk of, slow or perhaps prevent altogether the development of a cardiovascular disorder. When the agent is one that binds to cellular adhesion molecules and inhibits the ability of white blood cells to attach to such molecules, then the agent may be used prophylactically or may be used in acute circumstances, for example, post-myocardial infarction or post-angioplasty. It will be recognized when the agent is used in acute circumstances, it is used to prevent one or more medically undesirable results that typically flow from such adverse events. In the case of myocardial infarction, the agent can be used to limit injury to the cardiovascular tissue which develops as a result of the myocardial infarction and in the case of restenosis the agent can be used in amounts effective to inhibit, prevent or slow the reoccurrence of blockage. In either case, it is an amount sufficient to inhibit the infiltration of white blood cells and transmigration of white blood cells into the damaged tissue, which white blood cells can result in further damage and/or complications relating to the injury.

Generally, doses of active compounds would be from about 0.01 mg/kg per day to 1000 mg/kg per day. It is expected that doses ranging from 50-500 mg/kg will be suitable, preferably orally and in one or several administrations per day. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The anti-inflammatory agents, anti-Lp-PLA2 agents or statins may be combined, optionally, with a pharmaceutically-acceptable carrier. The term “pharmaceutically-acceptable carrier” as used herein means one or more compatible solid or liquid filler, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

Compositions suitable for parenteral administration conveniently comprise a sterile aqueous preparation of the anti-inflammatory agent, which is preferably isotonic with the blood of the recipient. This aqueous preparation may be formulated according to known methods using suitable dispersing or wetting agents and suspending agents The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

A variety of administration routes are available. The particular mode selected will depend, of course, upon the particular drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations. Oral administration will be preferred for prophylactic treatment because of the convenience to the patient as well as the dosing schedule.

The pharmaceutical compositions may conveniently be presented in unit dosage form and may be prepared by any of the methods well-known in the art of pharmacy. All methods include the step of bringing the anti-inflammatory agent into association with a carrier which constitutes one or more accessory ingredients. In general, the compositions are prepared by uniformly and intimately bringing the anti-inflammatory agent into association with a liquid carrier, a finely divided solid carrier, or both, and then, if necessary, shaping the product.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

An aspect of the invention comprises a method for detecting vascular disease in an individual comprising utilizing the methods described above to determine the individual's Lp-PLA2 activity in a sample wherein increased activity of Lp-PLA2 in the sample is indicative of vascular disease. In a preferred embodiment the vascular disease is selected from the group consisting of coronary vascular disease (CVD), coronary heart disease (CHD), peripheral vascular disease, peripheral arterial disease, high blood pressure, stroke, congenital cardiovascular defects and congestive heart failure.

Another aspect of the invention comprises a method for selecting a therapy to treat vascular disease for an individual comprising utilizing the methods described above to determine the individual's Lp-PLA2 level in a sample wherein increased activity or mass of Lp-PLA2 in the sample is indicative of an individual who will benefit from therapy to treat vascular disease. In particular, a low level of Lp-PLA2 indicates a subject likely to benefit from thrombolytic therapy while a high level of Lp-PLA2 indicates a subject likely to benefit from aggressive thrombolytic therapy, drug combinations and/or interventional and surgical approaches. In a preferred embodiment the vascular disease is selected from the group consisting of coronary vascular disease (CVD), coronary heart disease (CHD), peripheral vascular disease, peripheral arterial disease, high blood pressure, stroke, secondary stroke, congenital cardiovascular defects and congestive heart failure. In another preferred embodiment the therapy is selected from the group consisting of thrombolytic, niacin, statins and Lp-PLA2 inhibitors.

A further aspect of the invention comprises a method for monitoring an individual's response to therapy to treat vascular disease comprising utilizing the methods described above to determine the individual's Lp-PLA2 activity in a sample wherein decreased activity of Lp-PLA2 in the sample is indicative of an individual who is responding favorably to therapy to treat vascular disease. In a preferred embodiment the vascular disease is selected from the group consisting of coronary vascular disease (CVD), coronary heart disease (CHD), peripheral vascular disease, peripheral arterial disease, high blood pressure, stroke, secondary stroke, congenital cardiovascular defects and congestive heart failure. In another preferred embodiment the therapy is selected from the group consisting of thrombolytic, niacin, statins and Lp-PLA2 inhibitors.

Unless otherwise defined herein, medical, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press (1989) and Sambrook et al., Molecular Cloning: A Laboratory Manual, 3d ed., Cold Spring Harbor Press (2001); Ausubel et al., Current Protocols in Molecular Biology, Greene Publishing Associates (1992, and Supplements to 2000); Ausubel et al., Short Protocols in Molecular Biology: A Compendium of Methods from Current Protocols in Molecular Biology—4^{th} Ed., Wiley & Sons (1999); Harlow and Lane, Antibodies: A Laboratory Manual, Cold Spring Harbor Laboratory Press (1990); and Harlow and Lane, Using Antibodies: A Laboratory Manual, Cold Spring Harbor Laboratory Press (1999).

Methods to measure Lp-PLA2 levels are known in the art. For instance, a competition assay may be employed wherein an anti-Lp-PLA2 antibody is attached to a solid support and an allocated amount of a labeled Lp-PLA2 and a sample of interest are incubated with the solid support. The amount of labeled Lp-PLA2 attached to the solid support can be correlated to the quantity of Lp-PLA2 in the sample. These assays and variations therefore comprise a further embodiment of the present invention.

The methods described herein can further be utilized as prognostic assays to identify subjects having or at risk of developing a disease or disorder associated with increased or decreased expression levels of Lp-PLA2. The presence of higher (or lower) Lp-PLA2 levels as compared to normal human controls is diagnostic for the human patient being at risk for developing CVD. The effectiveness of therapeutic agents to decrease (or increase) expression or activity of Lp-PLA2 of the invention can also be monitored by analyzing levels of expression of the Lp-PLA2 in a human patient in clinical trials or in vitro screening assays such as in human cells. In this way, the gene expression pattern can serve as a marker, indicative of the physiological response of the human patient or cells, as the case may be, to the agent being tested.

The methods described herein can further be utilized in an acute care setting or timeframe.

The above tests can be carried out on samples derived from a variety of cells, bodily fluids and/or tissue extracts such as homogenates or solubilized tissue obtained from a patient. Tissue extracts are obtained routinely from tissue biopsy and autopsy material. Bodily fluids useful in the present invention include blood, urine, saliva or any other bodily secretion or derivative thereof. As used herein “blood” includes whole blood, plasma, serum, circulating epithelial cells, constituents, or any derivative of blood.

In addition to detection in bodily fluids, the proteins and nucleic acids of Lp-PLA2 are suitable to detection by cell capture technology. Whole cells may be captured by a variety of methods for example magnetic separation, U.S. Pat. Nos. 5,200,084; 5,186,827; 5,108,933; 4,925,788, the disclosures of which are incorporated herein by reference in their entireties. Epithelial cells may be captured using such products as Dynabeads® or CELLection™ (Dynal Biotech, Oslo, Norway). Alternatively, fractions of blood may be captured, e.g., the buffy coat fraction (50 mm cells isolated from 5 ml of blood) containing epithelial cells. Cells may also be captured using the techniques described in WO 00/47998, the disclosure of which is incorporated herein by reference in its entirety. Once the cells are captured or concentrated, the proteins or nucleic acids are detected by the means described in the subject application. Alternatively, nucleic acids may be captured directly from blood samples, see U.S. Pat. Nos. 6,156,504, 5,501,963; or WO 01/42504, the disclosures of which are incorporated herein by reference in their entireties.

### EXAMPLES

The present invention may be better understood by reference to the following nonlimiting examples.

### Example 1

### Introduction and Study Populations

Lp-PLA2 levels were evaluated in well phenotyped stroke cohorts from available stored samples. Informed consents were received from the patients for the study of blood biomarkers to permit prognostic studies.

Lp-PLA2 levels were evaluated in the “acute phase” of a stroke to evaluate temporal profile of Lp-PLA2 after stroke and to test prognostic value of Lp-PLA2 in the acute setting.

Lp-PLA2 levels were also evaluated in the “sub-acute phase” of a stroke to evaluate the utility of Lp-PLA2 to predict a second stroke (or any vascular event), predict a second stroke after transient ischemic attack (TIA), and predict a second stroke among specific stroke subtypes (i.e. atherosclerotic stroke due to intracranial stenosis).

### Acute Phase Study

The cohorts evaluated for the acute phase study were (i) 20 patients with blood draws at 4 time-points (80 samples) (to evaluate temporal profile of Lp-PLA2 after stroke) and (ii) 100 patients that had blood drawn within three hours of having a stroke (to test prognostic value of Lp-PLA2 in the acute setting). Similar protocols for this study have been previously described by Montaner J et al (Stroke. 2006; 37(5):1205-10) the disclosure of which is herein incorporated by reference in its entirety.

### Sub-Acute Phase Study

The cohorts evaluated for the sub-acute phase study were (i) 77 patients who had recurrent events after a stroke and 77 patients who did not have recurrent events after a stroke (to predict a second stroke), (ii) 135 patients who had a TIA (to predict a second stroke after TIA), and (iii) 135 patients who had a stroke that has been characterized by subtype (predict a second stroke among specific stroke subtypes). Similar protocols for these study have been previously described by Castillo J et al (J Neurol. 2009 February; 256(2):217-24), Purroy F et al (Acta Neurol Scand. 2007; 115(1):60-6), and Arenillas J F et al (Stroke. 2003; 34(10):2463-8), respectively, the disclosures of which are herein incorporated by reference in their entirety.

### Measurement of Lp-PLA2

Lp-PLA2 mass was assayed using the PLAC® Test (diaDexus, Inc.) and Lp-PLA2 activity was assayed using a colorimetric activity method (diaDexus, Inc.).

### Example 2

### Temporal Profile of Lp-PLA2 After Stroke (Acute Phase)

### Study Protocol and Results

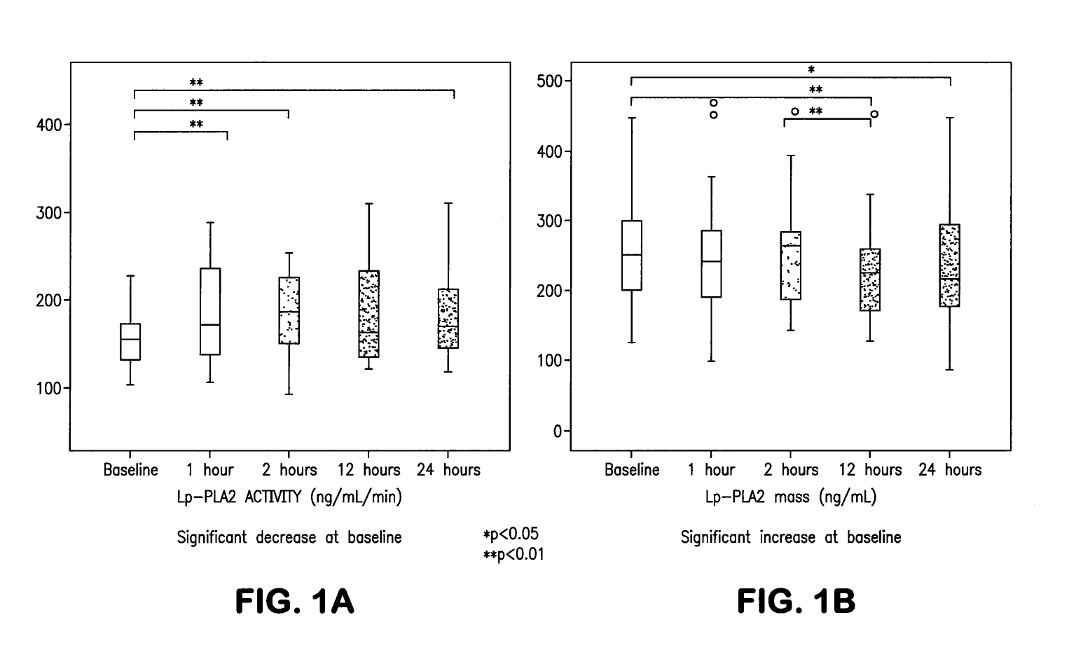

Peripheral blood samples were drawn at baseline (less than 3 hours from stroke onset) and serially thereafter. Specifically, in a series of 19 patients, blood samples were taken serially during the acute phase (baseline, 1 hour after (by the end of the tPA treatment), 2 hours after t-PA, and 12 and 24 hours after stroke onset. FIGS. 1A and 1B demonstrate that Lp-PLA2 activity levels were decreased at baseline compared to later time-points and Lp-PLA2 mass levels were increased at baseline compared to later time-points.

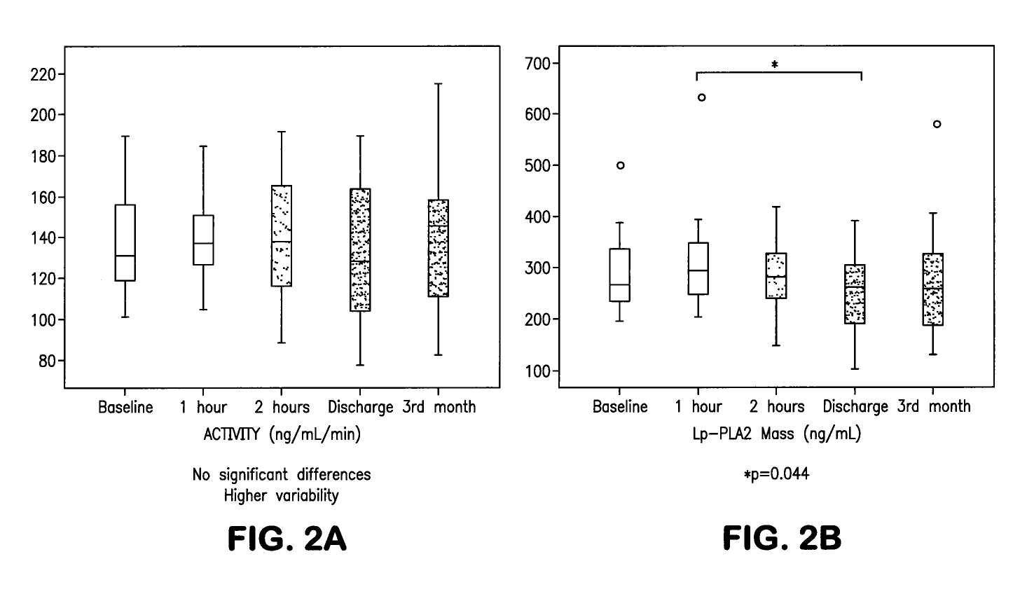

Also, in 15 patients, blood samples were obtained at baseline, 1 hour after (by the end oft-PA infusion), 24 hours after stroke onset, by discharge and at the third month visit. FIGS. 2A and 2B demonstrate that Lp-PLA2 mass significantly decreased between 1 hour after baseline and time of discharge. However, there was no significant difference between baseline and discharge or baseline and the 3 month time-point.

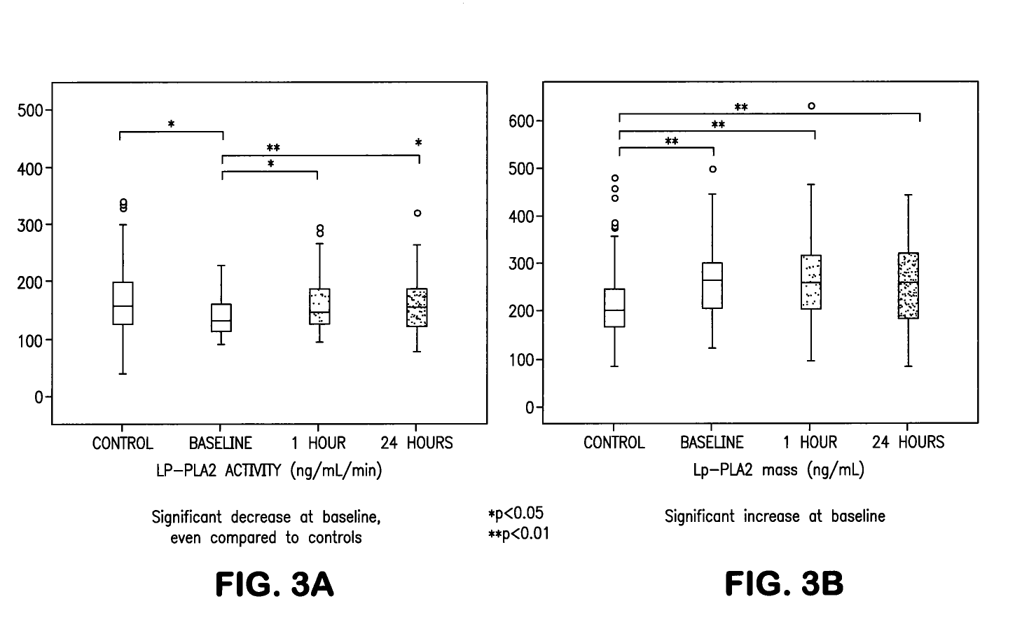

Finally, results from baseline, 1 hour and 24 hours in both groups were pooled (n=35 at each time point) and compared to a pooled control group (n=135). FIG. 3A demonstrates that Lp-PLA2 levels were significantly decreased at baseline in subjects having a stroke when compared to later time-points and controls. FIG. 3B also demonstrates that Lp-PLA2 mass was significantly increased in subjects with a stroke at baseline and later time-points compared to controls.

### Example 3

### Prognostic Value of Lp-PLA2 in the Acute Setting

### Study Protocol

Our study protocol included 100 consecutive stroke patients with a documented arterial occlusion who received thrombolytic treatment within the first 3 hours from symptoms onset. For the purpose of this study, only 92 patients with a middle cerebral artery (MCA) occlusion were analyzed.

A detailed history of vascular risk factors was obtained from each patient and to identify potential etiology of cerebral infarction, all patients underwent a set of diagnostic tests, including electrocardiogram, chest radiography, carotid ultrasonography, complete blood count and biochemistry.

Clinical examinations were performed on admission and at 12, 24 and 48 hours from symptom onset by means of National Health Institutes Stroke Scale (NIHSS) score. Neurological deterioration was defined as the increase of 4 or more points in NIHSS score between baseline and any other time point through follow-up. Likewise, neurological improvement was defined as the decrease in 4 or more points in that scale during the follow-up. Finally, functional outcome was defined according to the modified Rankin Scale (mRS) score. Patients scoring 3 or more points were considered to be functionally dependent.

To evaluate vessel status, transcranial Doppler measurements were performed before t-PA administration and serially (1, 2, 6 and 24 hours) thereafter, by an experienced neurologist using a Multi-Dop X4 (DWL Elektoniche Systeme GmbH, Sipplingen, Germany) device, with a hand-held transducer in a range-gated, pulsed-wave mode at a frequency of 2 MHz. On admission, the location of the MCA occlusion was recorded as proximal or distal. The presence of recanalization on follow-up TCD examinations was assessed according to the Thrombolysis in Brain Ischemia (TIBI) flow grading system. Complete recanalization was diagnosed as improvement to a stenotic or normal (TIBI 4 to 5 flow grades) waveforms; partial recanalization was diagnosed as an improvement in residual flow signals by at least 1 TIBI flow grade up to TIBI flow grades 2 to 3, and no recanalization was defined as the absence of improvement of the residual flow signal from baseline TCD.

Peripheral blood samples were drawn from each patient at baseline (before tPA administration and within 3 hours from onset).

### Results

Baseline Characteristics Associated with Lp-PLA2 Levels

All the factors associated with the baseline Lp-PLA2 mass and activity were determined. The results regarding demographics and vascular risk factors are shown in Table 1. Dyslipemic patients showed higher Lp-PLA2 activity than non-dyslipemic patients (182.2 vs 157.2, p=0.058) and also, patients who were taking statins had lower Lp-PLA2 mass concentrations than those patients who were not taking statins by the time of the stroke (244.5 versus 283, p=0.17).

TABLE 1
Demographic characteristics and vascular
risk factors associated with Lp-PLA2.

| | Lp-PLA2 mass | | | Lp-PLA2 activity | | |
|---|---|---|---|---|---|---|
| | (ng/mL) | | p | (ng/mL/min) | | p |
| All | 211 | (204.2-350.2) | — | 161.4 | (131.3-189.3) | — |
| Gender | | | | | | |
| Male | 292 | (236.5-352.2) | 0.56 | 162.9 | (136.1-198) | 0.62 |
| Female | 247 | (200.5-350) | | 161.6 | (122.3-161.6) | |
| Age | | | | | | |
| ≦76 | 277.5 | (201.7-347) | 0.78 | 157.6 | (130.6-185) | 0.53 |
| >76 | 256 | (202-390) | | 162.3 | (130-197.4) | |
| Tobacco | | | | | | |
| use | | | | | | |
| No | 259.5 | (201.3-350.7) | 0.17 | 162.4 | (133.4-197.9) | 0.26 |
| Yes | 288 | (271-367.5) | | 152.9 | (125.6-176.4) | |
| Hyper- | | | | | | |
| tension | | | | | | |
| No | 294 | (236.5-370.5) | 0.6 | 162.3 | (130-196.7) | 0.99 |
| Yes | 264 | (202-346) | | 161.6 | (131-189.3) | |
| Diabetes | | | | | | |
| No | 283 | (214-353) | 0.1 | 162 | (134-194.5) | 0.37 |
| Yes | 237 | (202-309.5) | | 158.4 | (119.4-195.5) | |
| Dyslipe- | | | | | | |
| mia | | | | | | |
| No | 275 | (209-349) | 0.68 | 157.2 | (127-181.7) | 0.058 |
| Yes | 279 | (198-380) | | 182.2 | (157.5-182) | |
| Previous | | | | | | |
| stroke | | | | | | |
| No | 272 | (202-349) | 0.79 | 161.6 | (130-194.7) | 0.76 |
| Yes | 331 | (215.5-386.5) | | 165.7 | (150.2-165.7) | |
| CAD | | | | | | |
| No | 261.5 | (203.7-350.7) | 0.64 | 161.4 | (133.4-186.8) | 0.85 |
| Yes | 296 | (232.5-361.5) | | 168.4 | (119.6-214.5) | |
| Anti- | | | | | | |
| platelet | | | | | | |
| No | 247 | (201.346) | 0.74 | 159 | (130.3-184.2) | 0.47 |
| Yes | 291 | (236-359) | | 165.9 | (134.6-165.9) | |
| Statins | | | | | | |
| No | 283 | (214-354) | 0.17 | 162.1 | (130.3-194.5) | 0.89 |
| Yes | 244.5 | (198-323.5) | | 162 | (132.1-195) | |

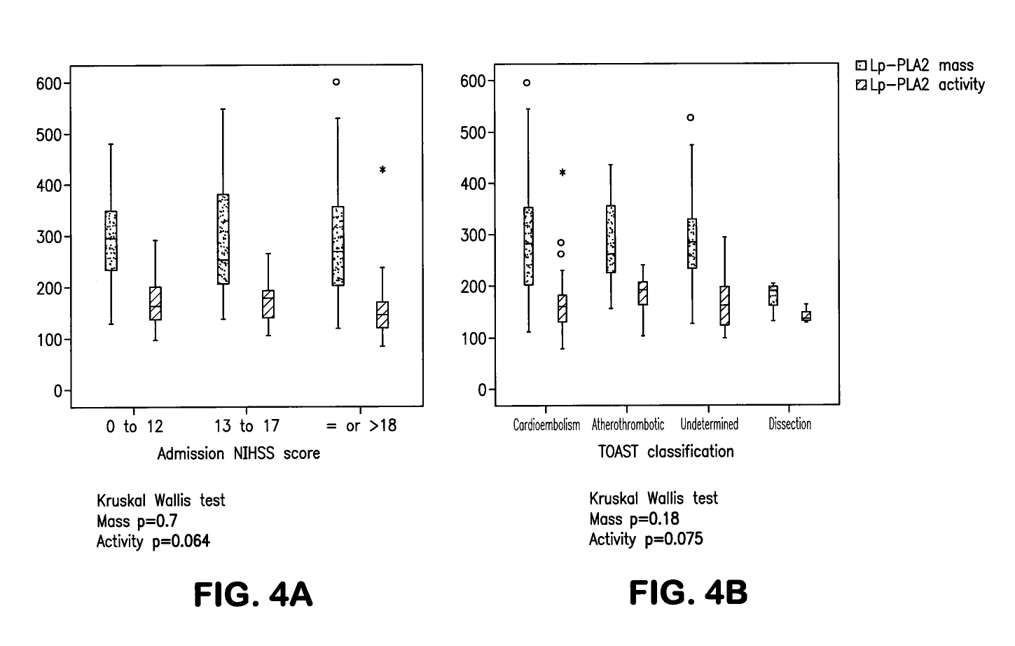

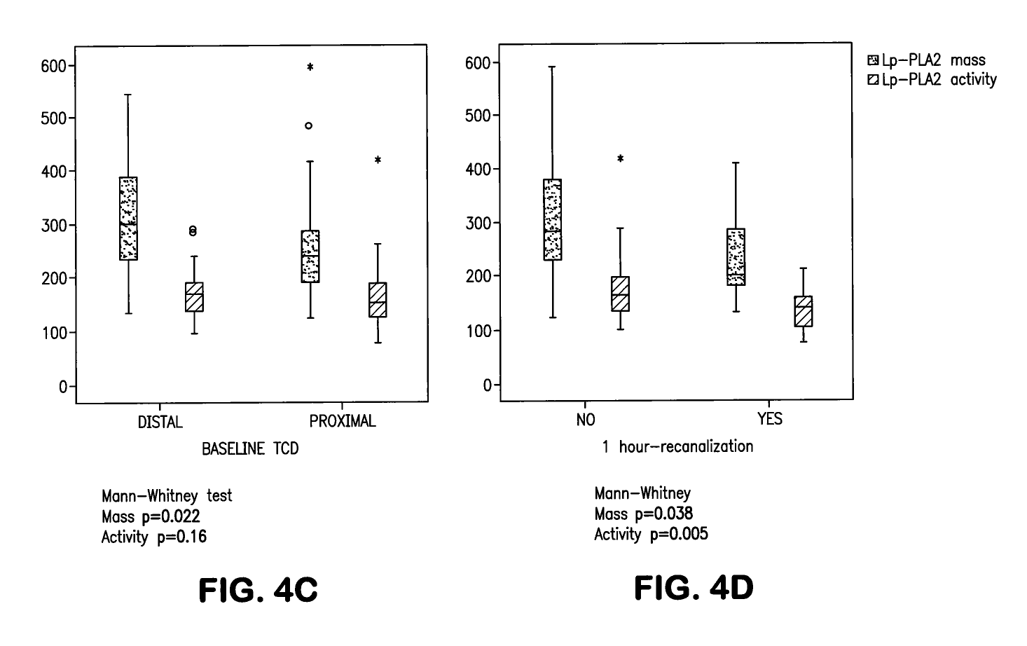

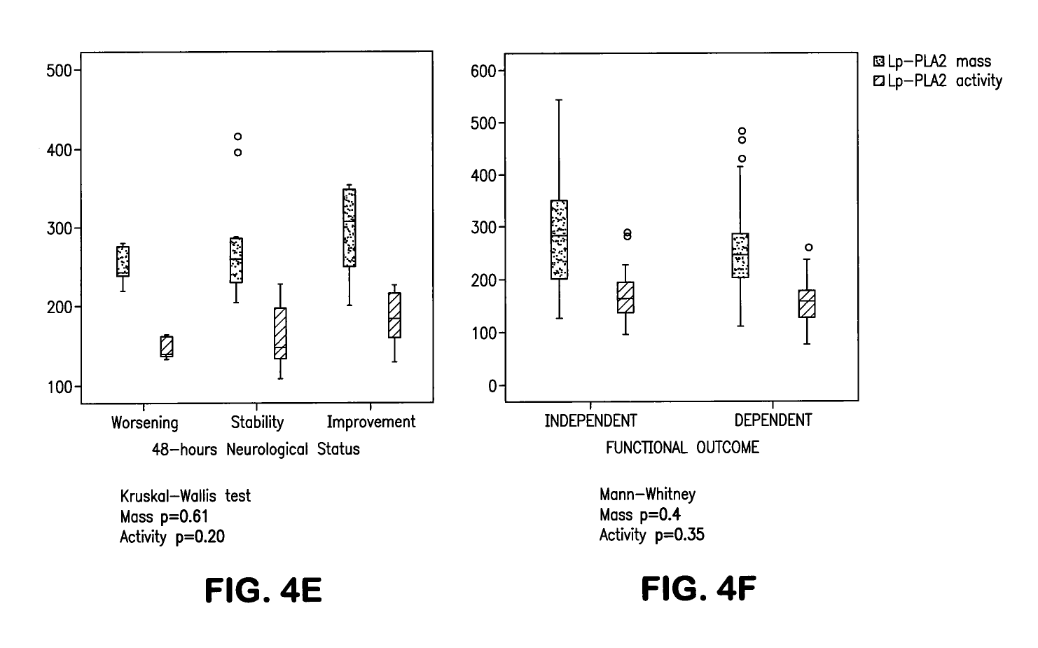

Also, associations between Lp-PLA2 mass and activity and stroke severity (measured by NIHSS score), etiology, as well as with the location of occlusion at baseline TCD (proximal versus distal occlusions) and the recanalization of the occluded vessel were studied (FIGS. 4A through 4F).

Lp-PLA2 activity and NIHSS score on admission showed a weak negative correlation (r=−0.23, p=0.032), with more severely affected patients showing the lowest Lp-PLA2 activity. Regarding etiology, the lowest mass and activity levels were found among patients with other determined stroke etiology, such as arterial dissection, and the highest levels in the patients with an atherothrombotic stroke etiology.

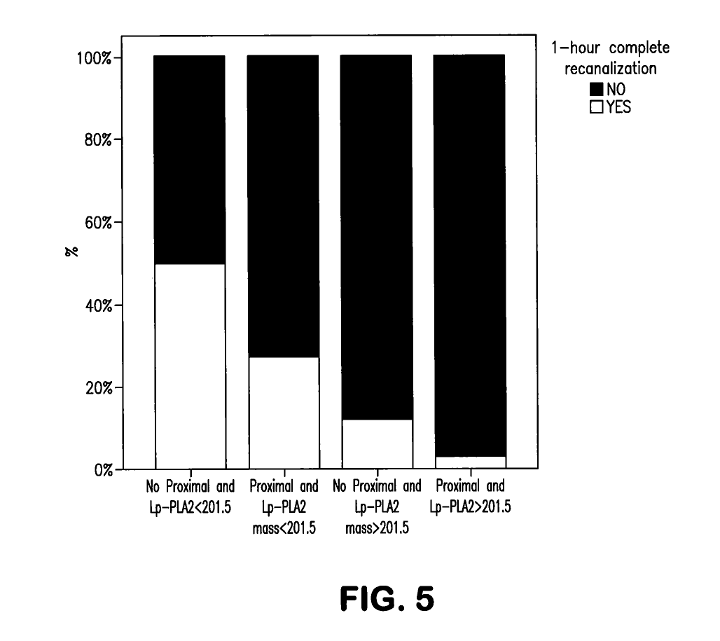

Regarding recanalization, both higher mass and activity levels were found among those patients who did not achieve complete recanalization by the end of thrombolytic therapy (tPA treatment), and after multivariate analysis (Table 2) adjusted by age, gender and vascular risk factors, Lp-PLA2 mass together with the presence of a proximal occlusion on baseline TCD were the most strongest predictors of occlusion persistence, and therefore resistance to thrombolytic treatment. Moreover, when a proximal occlusion was combined with Lp-PLA2 mass higher than 201.5 ng/mL, almost none of the patients achieved complete recanalization at 1 hour as compared with 50% of those without proximal occlusions and Lp-PLA2 mass lower than that level. (see FIG. 5; p for trend <0.001).

TABLE 2
Significant predictors for absence of complete recanalization
at the end of the tPA treatment after multivariate analyses.

| | OR | | p |
|---|---|---|---|
| Proximal occlusion on baseline TCD | 6.75 | (1.11-40) | 0.037 |
| Baseline Lp-PLA2 mass | 1.016 | (1.004-1.028) | 0.011 |
| Baseline Lp-PLA2 activity | 1.022 | (0.99-1.05) | 0.12 |

Adjusted by age, gender and vascular risk factors.

These results demonstrate that patients with high Lp-PLA2 levels did not benefit from thrombolytic therapy as well as those with low Lp-PLA2 levels. Therefore, patients with high Lp-PLA2 levels who are having a vascular event, or are suspected of having a vascular event, will benefit from more aggressive drug dosing (e.g. thrombolytic therapy) and drug combinations as well as interventional and surgical approaches.

Further, for patients with high Lp-PLA2 and a proximal occlusion, almost none benefit from thrombolytic therapy. Therefore, patients with a proximal occlusion and high Lp-PLA2 levels who are having a vascular event, or are suspected of having a vascular event, will benefit from more aggressive drug dosing (e.g. thrombolytic therapy) and drug combinations as well as interventional and surgical approaches.

Finally, neurological status during the acute phase of stroke and functional status at third month were explored and the results are shown in FIGS. 4E and 4F. No relation was found between Lp-PLA2 mass or activity and neurological status during the first 48 hours. Moreover, neither Lp-PLA2 mass or activity themselves were related with functional outcome at the third month.

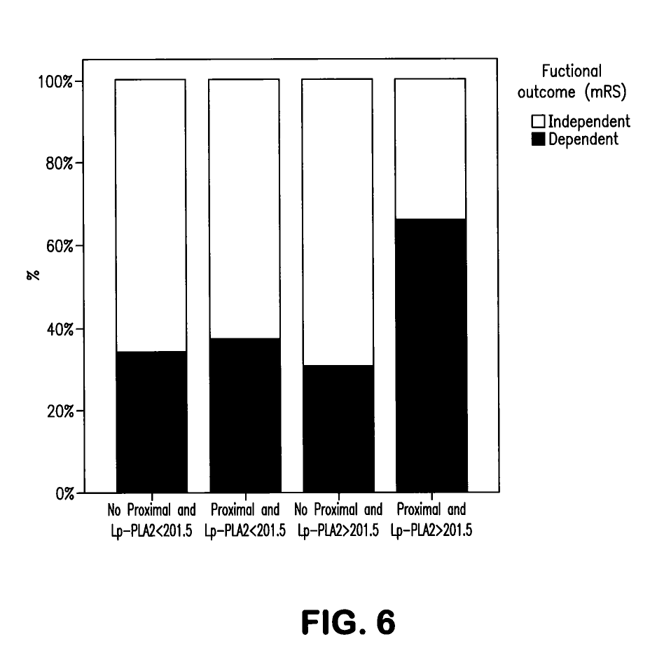

Lp-PLA2 levels add significant prognostic information when combined with the presence of a proximal occlusion. Lp-PLA2 levels and the presence/absence of a proximal occlusion predict a patient's 3 month functional outcome. Patients with high Lp-PLA2 levels (mass or activity) and a proximal occlusion are more likely to be functionally dependent at three months after a vascular event (FIG. 6). Patients who are more likely to be functionally dependent may benefit from additional or more aggressive therapies including thrombolytics (e.g. tPA) and surgical/PCI intervention.

### Example 4

### Lp-PLA2 Predicts a Second Stroke after TIA (or the Combined End-Point of Recurrent Non-Fatal Stroke, Non-Fatal Myocardial Infarction and Vascular Death)—Subacute Phase

### Study Protocol

In this case, we prospectively studied 166 consecutive patients with transient neurologic deficit attended by the neurologist in the emergency department. TIA was defined as a reversible episode of neurologic deficit of ischemic origin that resolved completely within 24 hours. A total of 11 clinical episodes were attributable to causes other than brain ischemia and were excluded for this study.

Demographics and classical vascular risk factors were recorded, as well as the clinical characteristics. Clinical symptoms and neurological signs at the examination were assessed and the vascular territory involved in each episode was recorded as carotid, vertebrobasilar or undetermined territory depending on the presence and combination of the above described findings. Finally, TIA duration and number of clinical episodes were recorded. TIA was categorized as single or a cluster of TIAs (when repeated TIAs occurred within the first week of the index event).

Blood samples for Lp-PLA2 determinations were drawn at the emergency department and always within the first 24 hours after the symptom onset.

Other examinations during admission included medical history, physical examination, routine blood biochemistry, electrocardiogram (ECG), chest X-ray, transthoracic echocardiography and Holter ECG when indicated, cervical carotid ultrasound and transcranial Doppler (TCD) ultrasonography; and CT scan. TCD recordings were performed on admission, within the first 24 h after symptom onset, with the use of a Multi-Dop-X/TCD device (DWL Elektronische Systeme GmbH; Compumedics Germany GmbH, Lindau, Germany). Intracranial stenoses were diagnosed if the mean blood flow velocity at a circumscribed insonation depth was >80 cm/s, with side-to-side differences>30 cm/s and signs of disturbed flow. Baseline cervical internal carotid artery (ICA) atherosclerosis was categorized by Eco Doppler as follows: absent; mild, if one or both ICAs had <50% stenoses; moderate, when any of the ICA presented <70% stenoses; and severe, if any ICA had >70% stenoses or there was a history of carotid surgery. Patients were classified as having large-artery occlusive disease if moderate or severe stenoses were detected by cervical and cranial ultrasonographic studies.

Once all the diagnostic tests had been performed, transient ischemic attacks were classified etiologically according to the Trial of ORG 10172(2) as due to large-artery occlusive disease (atherothrombotic), small-vessel disease, cardioembolic, uncommon or undetermined cause. Patients were followed up for 12 months and clinical interviews were performed at the seventh day, 1 month and every 3 months during the follow-up. End point events included further stroke or TIA, and the combined end-point of stroke, myocardial infarction or vascular death.

### Statistical Analyses

After the examination of the distribution was analyzed, Lp-PLA2 mass and activity were categorized by quartiles for further analysis. Cumulative event-free rates for the time to a stroke or any vascular event were estimated by the Kaplan-Meier product limit method, and patients with Lp-PLA2 mass and activity highest and lowest quartile were compared by the log-rank test.

Time to recurrent stroke or the combined end-point was analyzed with censoring at the time to either non-vascular death or last follow up.

Also, a receiver operating characteristic (ROC) curve was performed to identify an optimal cut-off point of LP-PLA2 activity that best discriminate between the presence or absence of a new recurrent event.

Cox proportional hazard models were constructed to estimate hazard ratios (HR) and 95% confidence intervals (95% CI) of the potential role of Lp-PLA2 as predictor of recurrent stroke, and the combined end-point of recurrent stroke, myocardial infarction, or vascular death after adjustment for age and classical vascular risk factors at the first week and the first month. P<0.05 was considered significant. Finally, because this was a post hoc analysis of a previously assembled cohort, power was not formally calculated prospectively.

### Results

### Baseline Characteristics and Lp-PLA2 Levels

Among the 166 TIA patients studied, 70% of them were first-ever stroke patients, whereas the remaining ones have had one or more previous strokes before the index event. The distribution of demographic factors, classical vascular risk factors and comorbid vascular diseases is given in Table 3. Regarding etiology, most of the TIA was of undetermined etiology after appropriate diagnostic tests were performed and affected the anterior circulation.

TABLE 3
Study participants characteristics

| Characteristic | Value | |
|---|---|---|
| Demographic characteristics | | |
| Age, mean ± SD, y | 74 | (66-81) |
| Gender, male | 86 | (52%) |
| Risk factors | | |
| Hypertension | 88 | (53%) |
| Diabetes mellitus | 41 | (25%) |
| Hyperlipemia | 44 | (27%) |
| Current smoking | 25 | (15%) |
| Alcohol | 7 | (4%) |
| Coronary artery disease | 25 | (15%) |
| Peripheral artery disease | 10 | (6%) |
| Atrial fibrillation | 38 | (23%) |
| Previous stroke | 48 | (30%) |
| Stroke etiology | | |
| Atherothrombotic | 38 | (23%) |
| Cardioembolic | 50 | (30%) |
| Small vessel occlusion | 7 | (4%) |
| Undetermined | 71 | (43%) |
| Previous treatments | | |
| Antiplatelet treatment | 53 | (32%) |
| Lipid-lowering treatment | 18 | (11%) |
| Oral anticoagulation | 18 | (11%) |

Data is expressed as mean ± SD or n (%) when appropriate

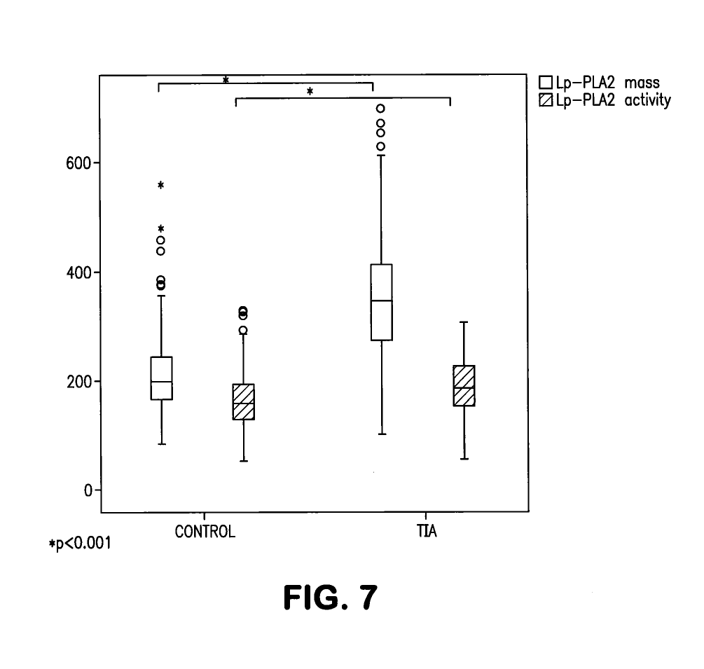

Lp-PLA2 mass and activity were not normally distributed in our population and both were significantly higher in TIA cases than in healthy controls ([347 (273 to 414)] vs [199 (167 to 243)], p<0.001 for Lp-PLA2 mass; [187 (151 to 228)] versus [160 (130 to 195)], p<0.001 for Lp-PLA2 activity), as it is shown in FIG. 7.

Among baseline characteristics and past medical history, several factors were found to be associated with Lp-PLA2 mass and/or activity (Table 4). Patients with past medical history of hyperlipemia had lower Lp-PLA2 mass and activity than patients without it and patients who were taking antiplatelets before the TIA also had lower Lp-PLA2 activity. There were no differences in the Lp-PLA2 level when other risk factors or previous treatments were considered.

TABLE 4
Demographic characteristics & vascular risk
factors according to Lp-PLA2 levels.

| | Lp-PLA2 Mass | P | Lp-PLA2 activity | P |
|---|---|---|---|---|
| Risk Factor | (ng/mL) | value | (ng/mL/min) | value |
| Overall (n = 166) | 347 (273-414) | — | 187 (151-228) | — |
| Age | | | | |
| <74 years (n=) | 390 (289-482) | 0.35 | 205 (158-237) | 0.54 |
| ≧74 years (n=) | 350 (264-416) | | 196 (167-240) | |
| Gender | | | | |
| Male (n = 86) | 352 (274-407) | 0.71 | 193 (161-232) | 0.067 |
| Female (n = 80) | 341 (272-433) | | 176 (142-221) | |
| Hypertension | | | | |
| No (n = 78) | 353 (275-419) | 0.57 | 187 (162-225) | 0.85 |
| Yes (n = 88) | 347 (266-434) | | 187 (143-235) | |
| Diabetes mellitus | | | | |
| No (n = 125) | 354 (277-434) | 0.47 | 188 (160-228) | 0.78 |
| Yes (n = 41) | 333 (264-392) | | 186 (145-236) | |
| Hyperlipemia | | | | |
| No (n = 122) | 360 (277-446) | 0.03 | 197 (277-446) | 0.007 |
| Yes (n = 44) | 316 (238-364) | | 167 (149-197) | |
| Smoking | | | | |
| No (n = 141) | 344 (269-411) | 0.67 | 187 (150-229) | 0.87 |
| Yes (n = 25) | 378 (294-448) | | 194 (158-215) | |
| Atrial fibrillation | | | | |
| No (n = 128) | 354 (268-419) | 0.8 | 184 (150-230) | 0.26 |
| Yes (n = 38) | 338 (276-432) | | 202 (161-228) | |
| Antiplatelet | | | | |
| No (n = 113) | 381 (277-482) | 0.18 | 213 (173-241) | 0.039 |
| Yes (n = 53) | 347 (259-398) | | 180 (148-233) | |
| Coronary artery | | | | |
| disease | | | | |
| No (n = 141) | 354 (276-435) | 0.095 | 191 (152-230) | 0.19 |
| Yes (n = 25) | 340 (264-361) | | 174 (149-206) | |
| Stroke etiology | | | | |
| Atherothrombotic | 389 (333-454) | 0.7 | 226 (175-298) | 0.11 |
| (n = 38) | | | | |
| Cardioembolic | 339 (268-410) | | 197 (150-226) | |
| (n = 50) | | | | |
| Small Vessel Occlu- | 372 (282-529) | | 179 (120-248) | |
| sion (n = 7) | | | | |
| Undetermined (n = 71) | 359 (266-456) | | 187 (155-235) | |

Data are expressed as median (interquartile range).

The presence of a documented large artery stenosis either at the intracranial circulation or intracranial plus extracranial vessels was associated with higher Lp-PLA2 activity.

### Follow-Up and Outcome Events

During follow-up, the presence of a new vascular events (coronary artery disease, stroke or TIA and peripheral vascular disease) was assessed early after the index TIA (during the first week) and later on at the first month and after 1-year follow-up. New vascular events were classified and recorded by an experienced neurologist as part of the outpatient clinic evaluation and by phone interviews. Table 5 shows the rate of new events according to the time of their verification. All vascular events during the first week were stroke/TIA.

TABLE 5
Rate of new vascular events depending
on the time they were assessed.

| Follow-up | Any vascular event | Stroke or TIA |
|---|---|---|
| 1 year | 41 (25%) | 33 (19.9%) |
| 1 month | 23 (13.9%) | 21 (12.7%) |
| 1 week | 9 (5.4%) | 9 (5.4%) |

Univariate analyses were performed to identify all the factors associated with the presence of stroke/TIA or any vascular event both within the first week and the first month. After ultrasonographic study, the atherothrombotic stroke etiology (i.e detection of an intracranial or extracranial stenosis responsible for the index event with no other cause for stroke) was the most important factor associated with the presence of a recurrent event (p<0.01 for all outcomes). Other baseline associated factors which showed p<0.1 in the univariate analyses were the past medical history of peripheral artery disease for any vascular event within the first week, and the past medical history of coronary artery disease, dislipemia and peripheral artery disease, when the event occurred within the first month.

### Outcome Events and ABCD 2

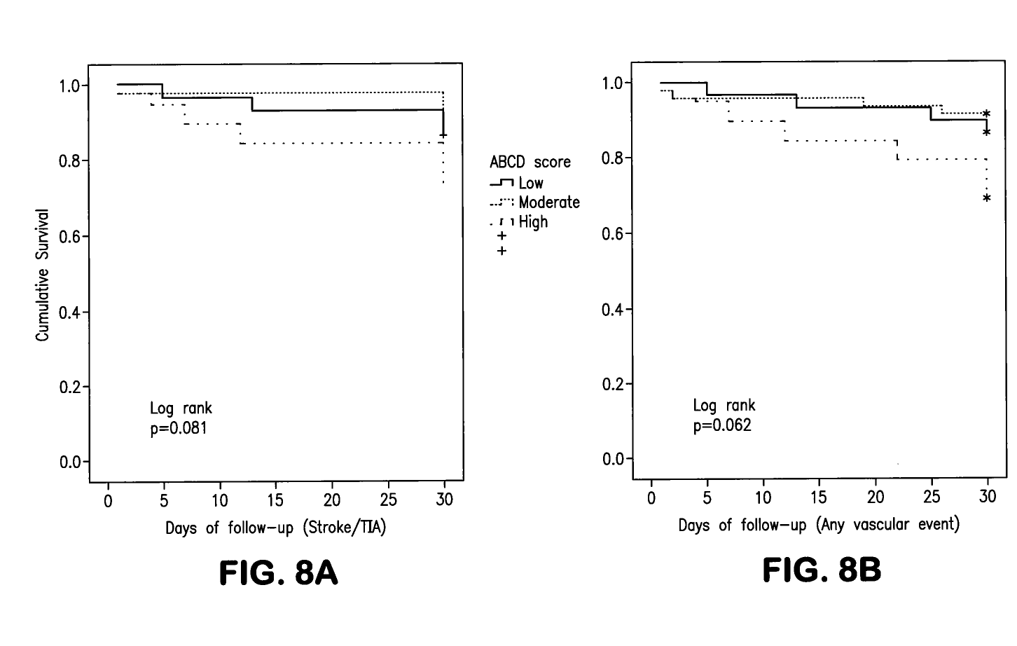

A subgroup of 96 patients with available clinical information for the index TIA event, were also classified according to the ABCD2 score to stratify the risk of further events. No significant differences were observed in the ABCD2 groups (low, moderate and intermediate risk) for the first week, and the obtained results regarding the first month are shown in FIGS. 8A and 8B. Patients classified as having high risk showed the maximum number of events, although not reaching statistical significance. Moreover, a considerable overlap in the low and moderate risk groups was observed regarding the number of events.

### Outcome Events and Lp-PLA2 Mass and Activity

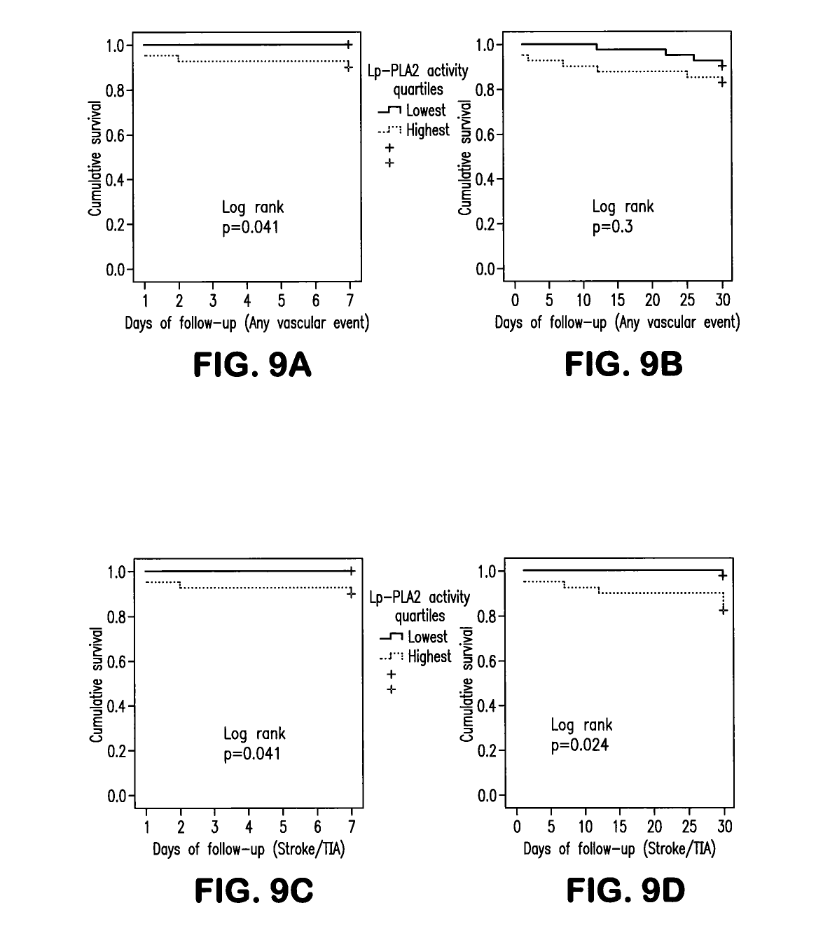

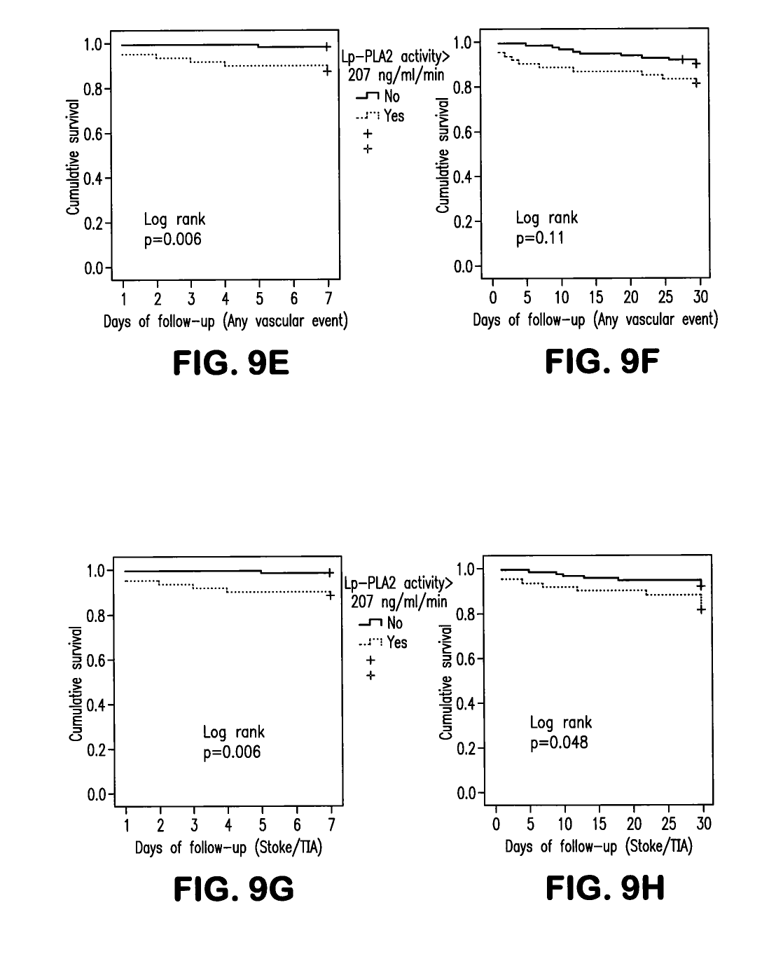

The relationship between Lp-PLA2 mass and activity with the presence or any further vascular event or stroke/TIA was analyzed by means of survival analyses. Patients at the lowest quartile of LP-PLA2 mass and activity versus those at the highest quartile were compared using the log-rank test. FIGS. 9A through 9D show the Kaplan-Meier curves regarding Lp-PLA2 activity quartiles. No significant differences were found in the rate of new events considering Lp-PLA2 mass quartiles (data not shown).

Finally, a receiver operating characteristic (ROC) curve identified an Lp-PLA2 activity of 207 ng/mL/min as the optimal cut-off point to discriminate the presence of a new (first week) stroke or TIA, with a 78% sensibility, 66% specificity (area under the curve equal to 0.71). The same cut-off point was used for all other outcomes. FIGS. 9E through 9H show Kaplan-Meier curves for the first week and month Stroke/TIA or any vascular event comparing groups above or below the 207 ng/mL/min cut-off point.

Finally, Cox regression models were performed to identify the potential predictors for the appearance of an early (first week) vascular event or late (1 month) stroke/TIA or vascular events and the results are shown in Table 6.

TABLE 6
Cox regression analyses to identify potential
predictors of early and late recurrent events.

| | HR (CI 95%) | p |
|---|---|---|
| Early Stroke/TIA or any vascular event | | |
| Atherothrombotic stroke etiology | 9.3 (1.81-48.2) | 0.008 |
| Late (1 month) Stroke/TIA | | |
| Dislipemia | 3.68 (1.04-7.07) | 0.008 |
| Atherothrombotic etiology | 3.28 (1.32-8.15) | 0.011 |
| Lp-PLA2 activity >207 ng/mL/min | 2.7 (1.04-7.07) | 0.042 |
| Late (1-month) vascular event | | |
| Atherothrombotic stroke etiology | 3.33 (1.39-7.94) | 0.007 |
| Coronary artery disease | 3.38 (1.36-8.41) | 0.009 |

All models are adjusted by age, gender, vascular risk factors and factors showing p < 0.1 in the univariate analyses.

### Example 5

### Lp-PAL2 Predict a Second Stroke or the Combined End-Point—Subacute Phase

The combined End-Point is recurrent non-fatal stroke, non-fatal myocardial infarction and vascular death in specific stroke subtype (atherosclerotic stroke due to intracranial stenoses).

### Patients and Methods

### Study Participants

Between June 2001 and January 2004, 196 consecutive patients with TIA or ischemic stroke admitted to our Stroke Unit showing intracranial stenoses potentially responsible for the cerebral ischemic event on TCD recordings were evaluated.

Diagnostic protocol examinations during admission included medical history, physical examination, routine blood biochemistry and blood count, EKG, chest x-ray, thyroid function, immunologic study, transthoracic echocardiography and EKG Holter when indicated, cranial MRI or CT scan including angiographic sequences, and cervical carotid ultrasound.

Included in this study were 75 consecutive patients in whom an angiographic confirmation of intracranial stenoses could be achieved by MR-angiography or CT-angiography. The same cohort has been described in detail in previous studies by our group (Arenillas J F (2008) Stroke 39: 1456-1463; Juan F (2009) Cerebrovasc Dis 28:95-102).

Reasons for excluding the remaining candidates belong to the following categories: (1) presence of other potential causes of cerebral ischemia (n=46), (2) non-atherosclerotic origin of intracranial stenoses (n=23), (3) existence of inflammatory conditions (n=20) and (4) impossibility of performing follow-up due to stroke-related death or severe disability (n=10), lack of an adequate acoustic window (n=21) or denial of informed consent (n=1). At the inclusion visit, which took place at least 3 months after the qualifying event, informed consent and blood samples were obtained from all 75 patients with symptomatic intracranial atherostenoses.

Blood samples were drawn always after overnight fast. Acute infections, surgery or trauma during the previous 3 months and incident neoplasm or inflammatory conditions were ruled out by a careful medical history and physical examination prior to sampling.

135 samples obtained from healthy volunteers and patients relatives were used as controls for this study.

This study was approved by the local ethics committee.

### Clinical Variables and Long-Term Follow-Up

Cigarette smoking and medical history of hypertension, hypercholesterolemia and type 2 diabetes mellitus were recorded at the inclusion visit. Stroke severity was assessed using the maximum National Institutes of Health Stroke Scale score during admission. Functional status at day 90 was assessed by means of the modified Rankin scale score (mRS). Secondary prevention therapies were established following the recommendations of the American Heart Association guidelines available during the study period. Antithrombotic treatment was indicated in an individualized manner following the criteria of the stroke team responsible for each patient. The use of acenocumarol, aspirin, clopidogrel, statins, angiotensin-converting enzyme inhibitors and angiotensin receptor blockers was registered.

After inclusion, clinical visits were conducted every 6 months by a stroke neurologist (J.F.A.) who remained unaware of the biochemical data of the patients throughout the study period. The following major vascular events were considered as predefined clinical end points: acute ischemic stroke; TIA diagnosed by a stroke neurologist; acute myocardial infarction or angina requiring hospitalization, and vascular death.

### Ultrasound Protocol

TCD recordings were performed using a Multi-Dop-X/TCD (DWL Elektronische Systeme GmbH, Germany) device, with a hand-held transducer in a range-gated, pulsed-wave mode at a frequency of 2 MHz. We used a standard method of insonation through the temporal, occipital, and orbital windows without compression testing, as previously described. According to validated criteria, intracranial stenoses were diagnosed if the mean blood flow velocity at a circumscribed insonation depth was >80 cm/s, with side-to-side differences of >30 cm/s and signs of disturbed flow. TCD examinations were carried out on admission and repeated at the inclusion visit to confirm the persistence of stenoses. Baseline cervical ICA atherosclerosis was categorized as absent; mild, if one or both ICAs had a mild <50% stenosis; moderate, when any of the ICAs presented a moderate <70% stenosis; and severe, if any ICA had a severe asymptomatic stenosis.

### Lp-PLA2 Mass and Activity Determinations

Total cholesterol high-density lipoprotein (HDL)-cholesterol, and low density lipoprotein (LDL)-cholesterol levels were determined by automatic enzymatic methods in serum samples.

Lp-PLA2 mass and activity were determined in EDTA-plasma samples by means of the PLAC test at an automated Olympus analyzer and by a colorimetric activity method (diaDexus). All samples were run in duplicates.

### Statistical Analysis

Analyses were performed with the SPSS statistical package (Chicago, Ill., USA), version 15.0. Statistical significance for intergroup differences was assessed by the X2 test or Fisher's exact test for categorical variables and by the Mann-Whitney U test for continuous variables.

To prevent overmodeling of the data and false-positive results, only clinical recurrence was considered an end point of the study.

Univariate analyses were performed to detect variables associated with Lp-PLA2 mass and activity as well as the occurrence of further ischemic events.

A Cox proportional hazards multivariate analysis was used to identify predictors of further ischemic events during follow-up, in which age, sex, current smoking, hypertension, diabetes, hypercholesterolemia and variables showing p values <0.05 on univariate testing were included. Results were expressed as adjusted hazard ratios and corresponding 95% confidence intervals.

### Results

### Baseline Clinical Variables

Baseline characteristics and vascular risk factors of the study population are shown in Table 7. The study sample consisted of 55 men (73%) and 20 women (27%). Mean age was 66.2±8.3 years. The qualifying event attributable to a symptomatic intracranial atherostenosis was an ischemic stroke in 54 patients (72%) and a TIA in the remaining 21 (28%). The symptomatic lesion was located in the intracranial internal carotid artery in 17 patients (23%), in the middle cerebral artery in 25 (33%), in the anterior cerebral artery in 2 (3%), in the posterior cerebral artery in 9 (12%) and in the vertebrobasilar system in 14 (19%). In 8 patients with multiple stenoses presenting with a TIA, it was not possible to determine which intracranial stenosis had been symptomatic. Very early recurrence during admission was observed in 26 patients; ischemic strokes were preceded by a TIA in 12 patients, and 14 patients presented with repeated TIAs.

For stroke patients, the median National Institutes of Health Stroke Scale score on admission was 2 (interquartile range 0-4). All studied subjects remained free of ischemic events during the period between hospital discharge and the inclusion visit. Regarding secondary prevention therapies, 58 patients (77%) received antiplatelet agents, 17 (23%) received oral anticoagulants and 53 (71%) were treated with statins throughout the follow-up period. Intracranial stenoses were confirmed by MRA in 66 patients (88%) and by CTA in 9 (12%). Besides the 75 symptomatic stenoses, a total of 165 coexistent asymptomatic stenoses were detected. The median number of intracranial stenoses per patient was 3 (interquartile range 2-4).

TABLE 7
Baseline characteristics of the study sample (n = 75).

| Characteristic | Value |
|---|---|
| Demographic characteristics | |
| Age, years | 66.2 ± 8.3 |
| Gender, male | 55 (73%) |
| Risk factors and comorbid | |
| vascular diseases | |
| Current smoking | 35 (47%) |
| Hypertension | 60 (80%) |
| Diabetes mellitus | 40 (53%) |
| Hypercholesterolemia | 55 (73%) |
| Coronary heart disease | 13 (17.5%) |
| Peripheral arterial disease | 15 (20%) |
| >2 vascular risk factors | 36 (48%) |
| Qualifying event | |
| Stroke | 54 (72%) |
| TIA | 21 (28%) |
| Location of symptomatic | |
| intracranial stenosis | |
| Intracranial ICA | 17 (23%) |
| MCA | 25 (33%) |
| ACA | 2 (3%) |
| PCA | 9 (12%) |
| VB | 14 (19%) |
| Undetermined | 8 (10%) |
| Asymptomatic extracranial | |
| ICA >30% stenosis | 28 (37%) |
| 90-day mRS score 0 or 1 | 62 (83%) |
| Antithrombotic treatment | |
| Anticoagulants | 17 (23%) |
| Aspirin | 26 (35%) |
| Clopidogrel | 43 (57%) |
| Statins | 53 (71%) |
| ACEI | 25 (33%) |
| ARB | 10 (13%) |

Results are expressed as means ± standard deviation, n (percentage) or medians (interquartile range) as appropriate. ACA = Anterior cerebral artery; ACEI = angiotensin converting enzyme inhibitors; ARB = angiotensin receptor blocker; ICA = internal carotid artery; MCA = middle cerebral artery; mRS = modified Rankin scale; PCA = posterior cerebral artery; VB = intracranial vertebral and basilar arteries.

### Lp-PLA2 Mass and Activity

Lp-PLA2 mass level was significantly higher in patients with symptomatic intracranial stenosis than in controls subjects (312.87 vs. 221.24 ng/ml, p<0.001). Regarding activity, it was significantly lower in cases than in controls (152.11 vs. 168.58, p=0.008). Decreased activity in cases could be related with the fact that most of the patients were under statins and antiplatelet treatments, which might have affected Lp-PLA2 activity measured three months after the ischemic event.

Table 8 shows univariate analyses regarding Lp-PLA2 mass and activity. In summary, mass was found higher in patients with an abnormal ankle-brachial index (ABI<0.9) and lower in patients under statins or clopidogrel treatments.

Regarding Lp-PLA2 activity, men had higher activity than women and patients with an abnormal ABI and with multiple or bilateral stenoses had also higher activity than patients with a single or unilateral stenosis.

Finally, patients with the lowest LDL-cholesterol levels (less than 100 mgrs/dL) or under statins had lower Lp-PLA2 activity than those without them.

TABLE 8
Demographic characteristics and vascular risk factors according to
Lp-PLA2 levels in patients with symptomatic intracranial stenosis.

| | Lp-PLA2 | | Lp-PLA2 | |
|---|---|---|---|---|
| | mass | | activity | |
| | (ng/ml) | P | (ng/ml/min) | P |
| Variable | Mean ± SD | value | Mean ± SD | value |
| Cases vs. controls | | | | |
| Cases | 312.87 ± 99.67 | <0.001 | 152.11 ± 36.58 | 0.008 |
| Controls | 221.24 ± 111.83 | | 168.58 ± 51.33 | |
| Gender | | | | |
| Men | 312.24 ± 99.65 | 0.951 | 158.09 ± 31.91 | 0.02 |
| Women | 314.55 ± 102.29 | | 135.97 ± 43.89 | |
| Age | | | | |
| <=66 years | 315.89 ± 108.41 | 0.953 | 146.05 ± 40.77 | 0.167 |
| >66 years | 310.00 ± 92.00. | | 157.86 ± 31.60 | |
| Current smoking | | | | |
| No | 303.59 ± 90.39 | 0.562 | 147.41 ± 40.20 | 0.246 |
| Yes | 323.20 ± 109.49 | | 157.35 ± 31.84 | |
| HTA | | | | |
| No | 311.53 ± 117.73 | 0.732 | 157.25 ± 34.92 | 0.546 |
| Yes | 313.20 ± 95.69 | | 150.81 ± 37.17 | |
| DM | | | | |
| No | 307.14 ± 105.74 | 0.398 | 147.53 ± 32.70 | 0.324 |
| Yes | 317.72 ± 95.29 | | 156.01 ± 39.58 | |
| DL | | | | |
| No | 327.89 ± 113.55 | 0.569 | 157.24 ± 44.72 | 0.482 |
| Yes | 307.67 ± 95.00 | | 150.34 ± 33.62 | |
| Basal LDL <100 | | | | |
| mg/dL | | | | |
| No | 314.07 ± 94.46 | 0.605 | 162.66 ± 34.28 | 0.002 |
| Yes | 311.10 ± 108.50 | | 136.64 ± 34.78 | |
| Coronary artery | | | | |
| disease | | | | |
| No | 317.11 ± 98.26 | 0.498 | 152.23 ± 36.11 | 0.743 |
| Yes | 298.67 ± 110.54 | | 156.02 ± 38.57 | |
| CV risk factors | | | | |
| 0-2 | 304.26 ± 101.02 | 0.317 | 149.31 ± 38.20 | 0.501 |
| >2 | 321.94 ± 98.82 | | 155.08 ± 35.09 | |
| TIA | | | | |
| Single | 295.14 ± 72.04 | 0.97 | 161.86 ± 41.43 | 0.19 |
| Repeated. | 325.79 ± 133.58 | | 136.90 ± 38.94 | |
| Stroke preceded by | | | | |
| TIA/repeated TIAs | | | | |
| No | 307.34 ± 90.15 | 0.457 | 155.29 ± 39.82 | 0.212 |
| Yes | 336.32 ± 115.37 | | 142.35 ± 35.31 | |
| Abnormal ABI | | | | |
| (<0.9) | | | | |
| No | 286.05 ± 112.41 | 0.032 | 135.85 ± 36.93 | 0.013 |
| Yes. | 327.08 ± 93.07 | | 159.37 ± 35.08 | |
| Carotídean lesion | | | | |
| No | 317.98 ± 100.63 | 0.612 | 151.62 ± 34.25 | 0.884 |
| Yes | 304.46 ± 99.32 | | 152.92 ± 40.78 | |
| Intracraneal stenosis | | | | |
| by TCD. | | | | |
| Unilateral. | 304.26 ± 126.06 | 0.172 | 139.82 ± 37.02 | 0.051 |
| Bilateral (basilar = | 316.74 ± 86.38 | | 157.66 ± 35.36 | |
| bilateral). | | | | |
| Intracraneal stenosis | | | | |
| by TCD. | | | | |
| Single | 291.64 ± 122.99 | 0.176 | 131.96 ± 27.40 | 0.047 |
| Multiple. | 316.57 ± 95.72 | | 155.63 ± 37.01 | |
| DTC progresion | | | | |
| No | 311.65 ± 99.78 | 0.841 | 150.50 ± 35.36 | 0.598 |
| Yes | 315.24 ± 101.46 | | 155.28 ± 39.43 | |
| Carotid atero- | | | | |
| sclerosis progresión | | | | |
| by Doppler | | | | |
| No | 317.56 ± 101.20 | 0.147 | 151.68 ± 37.28 | 0.710 |
| Yes | 248.00 ± 39.41 | | 158.04 ± 27.39 | |
| N° of lesions in | | | | |
| DWI | | | | |
| Single | 311.21 ± 99.03 | 0.183 | 152.77 ± 38.67 | 0.681 |
| Multiple | 353.18 ± 103.84 | | 157.51 ± 35.44 | |
| Leucoaraiosis | | | | |
| No | 315.67 ± 92.49 | 0.281 | 154.43 ± 35.24 | 0.537 |
| Yes. | 347.00 ± 98.66 | | 160.83 ± 37.31 | |
| Chronic lacunar | | | | |
| infarcts in RM | | | | |
| No | 320.65 ± 100.97 | 0.578 | 159.58 ± 37.84 | 0.780 |
| Yes. | 333.77 ± 99.31 | | 156.70 ± 36.54 | |
| Microhemorrages in | | | | |
| RM | | | | |
| No | 322.86 ± 93.34 | 0.664 | 157.45 ± 34.72 | 0.569 |
| Yes. | 311.53 ± 87.61 | | 151.17 ± 38.07 | |
| Secondary preven- | | | | |
| tion until basal | | | | |
| extraction: | | | | |
| Statins | | | | |
| No | 333.44 ± 90.65 | 0.034 | 161.16 ± 35.72 | 0.049 |
| Yes | 295.37 ± 104.68 | | 144.42 ± 35.97 | |
| IECAs | | | | |
| No | 318.22 ± 101.46 | 0.544 | 153.05 ± 35.83 | 0.746 |
| Yes | 301.00 ± 96.72 | | 150.04 ± 38.94 | |
| Preventive treat- | | | | |
| ment. | | | | |
| Antiplaquelets | 300.68 ± 99.10 | 0.082 | 152.18 ± 34.05 | 0.981 |
| Dicumarinics. | 338.25 ± 98.03 | | 151.97 ± 42.16 | |
| Aspirin | | | | |
| No | 310.20 ± 99.94 | 0.648 | 151.20 ± 38.54 | 0.758 |
| Yes | 318.42 ± 101.12 | | 154.02 ± 32.82 | |
| Triflusal | | | | |
| No | 317.59 ± 101.87 | 0.195 | 153.41 ± 36.64 | 0.309 |
| Yes | 259.33 ± 47.38 | | 137.46 ± 35.70 | |
| Clopidogrel | | | | |
| No | 343.51 ± 96.45 | 0.001 | 156.62 ± 39.03 | 0.240 |
| Yes | 274.79 ± 91.30 | | 146.51 ± 33.03 | |
| Basal episode: | | | | |
| TIA | 326.30 ± 111.08 | 0.647 | 152.70 ± 40.26 | 0.926 |
| Established infarct. | 308.96 ± 96.33 | | 151.79 ± 35.86 | |

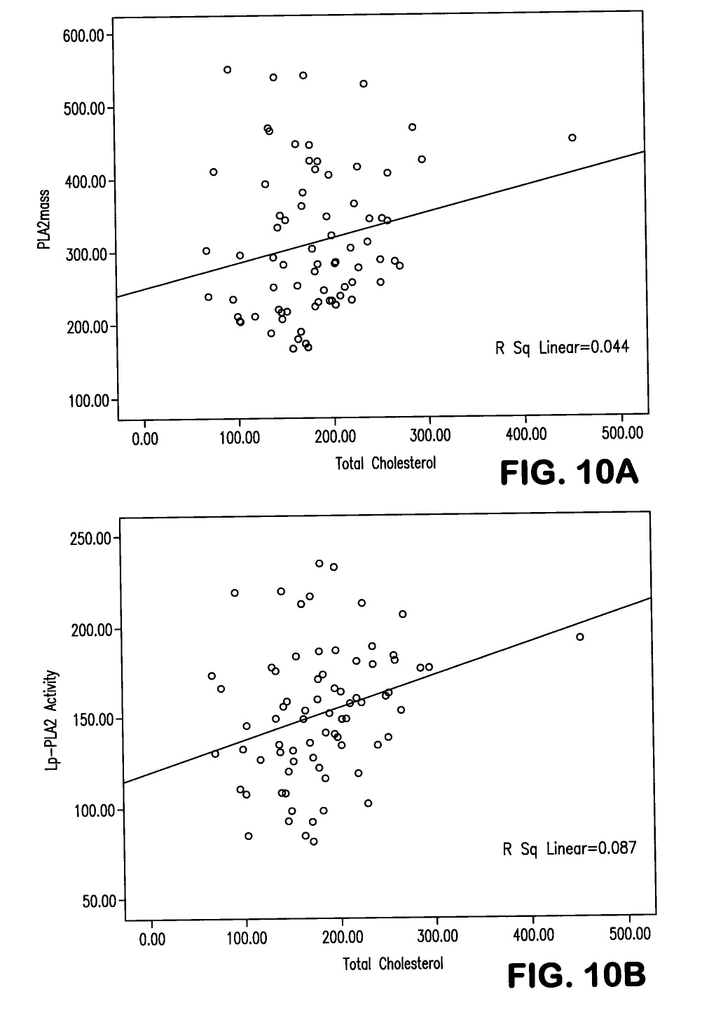

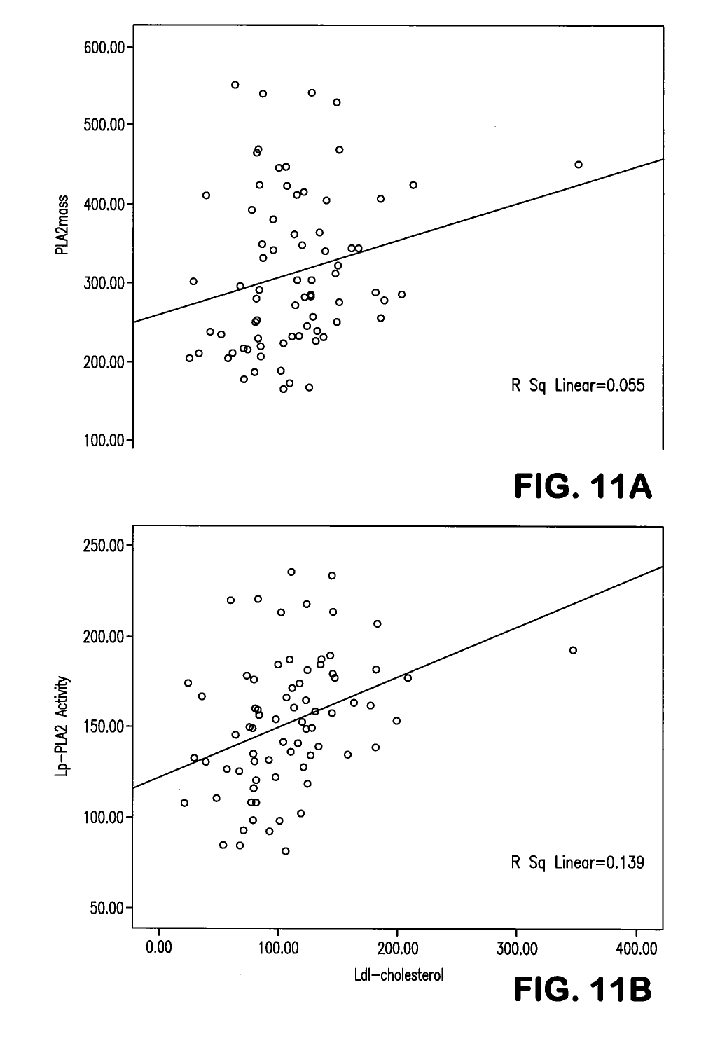

Also, several significant correlations were found between Lp-PLA2 mass and activity and total cholesterol. Correl. coe-0.225, p=0.054 for mass and Correl. coef.=0.294, p=0.011, for activity, see FIGS. 10A and 10B. LDL-cholesterol (Correl. coef.=0.372, p=0.001 for mass and Correl. coef=0.278, p=0.016, for activity, see FIGS. 11A and 11B.

### Example 6

### Lp-PAL2 Predicts of New Major Vascular Events—Subacute Phase

During a median follow-up time of 23 months (interquartile range 17-29), 18 patients (24%) suffered a major ischemic event, categorized as follows: 10 ischemic strokes, 3 TIAs and 5 myocardial infarctions. Of these major ischemic events, 9 (50%) occurred within the first 5 months after inclusion and only 4 took place after the first year of follow-up.

All recurrent cerebral ischemic events were attributable to intracranial atherostenoses, located as follows: in the intracranial internal carotid artery in 5 patients, in the middle cerebral artery in 5, in the basilar artery in 2 and in the posterior cerebral artery in 1. Three patients died during follow-up, 2 due to fatal strokes and 1 due to cancer.

Several factors were significantly associated with the pre-specified combined end-point (stroke/TIA, MI/angina or vascular death) in the survival analyses, such as the past medical history of coronary artery disease (p=0.046), the number of stenoses detected by TCD (p=0.007), progression of intracranial stenoses detected by TCD over time (p<0.001), the presence of an abnormal ankle-brachial index (ABI<0.9) (p=0.002) and an increased Lp-PLA2 activity.

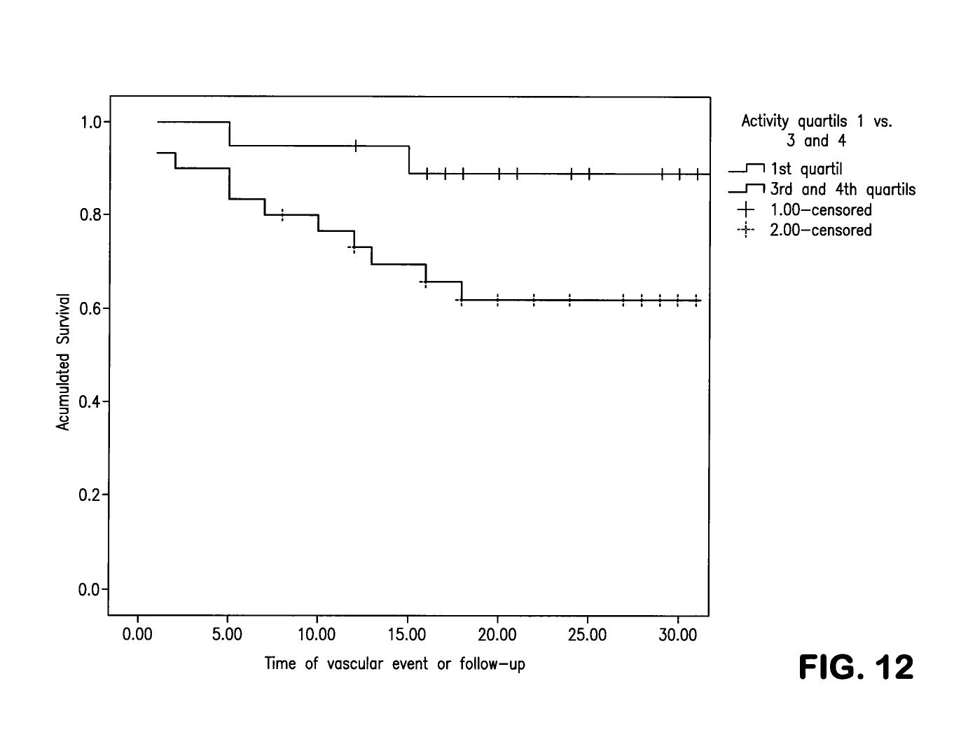

Regarding Lp-PLA2 activity across quartiles, we observed a significantly higher rate of events in the patients at the highest quartiles (3rd and 4Th), compared with the patients at that the lowest quartile (FIG. 12).

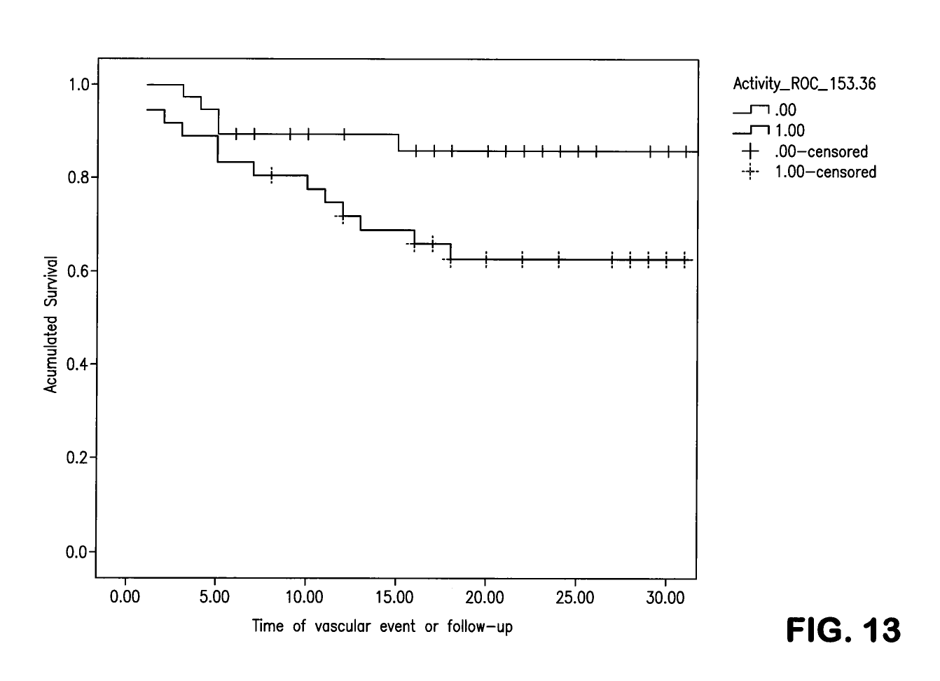

Also, a ROC curve identified 153.36 nmol/mL/min as an optimal cut-off point (sensitivity 0.72 and specificity 0.59) to discriminate between the patients who experienced a recurrent vascular event and the patients who did not. FIG. 13 shows the Kaplan-Meier curve considering the vascular events in the groups with Lp-PLA2 above and below this cut-off point.

Finally, in order to find potential predictors of recurrent vascular events, a multivariate analysis (Cox Regression) was performed including baseline clinical variables and Lp-PLA2 activity. Among all them, Lp-PLA2 activity higher than 153.36 nmol/mL/min was the strongest predictor of vascular recurrence (HR 2.89 (1.029-8.096), p=0.044).

Therefore, measurement of Lp-PLA2 activity might be potentially useful in the daily clinics evaluation of vascular recurrence risk, especially in patients without other available instrumental data (such as TCD or ABI).

We have also shown that Lp-PLA2 activity is not related to DTC progression. Therefore, Lp-PLA2 activity might be associated with vascular recurrence by mechanisms other than atherosclerosis progression (i.e. plaque instability).

Another clinical scenario could include the results of other complementary tests, such as ABI, which as it was shown before is also associated with vascular recurrence.

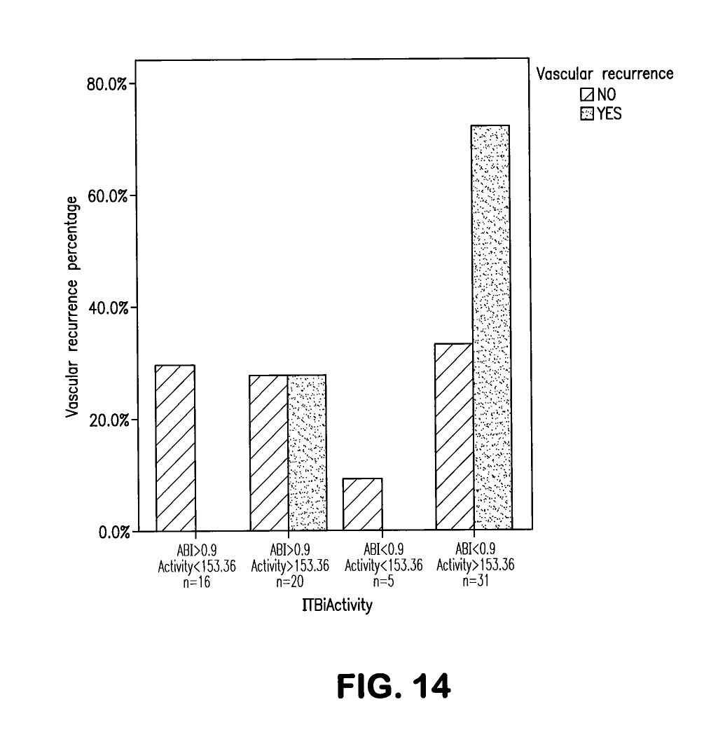

Then, we explored how the combination between Lp-PLA2 activity and ABI may improve the risk stratification, and the results are shown in FIG. 14.

High levels of Lp-PLA2 activity increase the risk of vascular recurrence among patients with an abnormal ABI (<0.9) although the same does not occur in patients with normal ABI.

All publications and other materials described herein are used to illuminate the invention or provide additional details respecting the practice and are incorporated by reference in their entirety.

1. A method of selecting a thrombolytic therapy for a subject, said method comprising determining a level of Lipoprotein-associated Phospholipase A2 (Lp-PLA2) in the subject;
wherein a low level of Lp-PLA2 indicates a subject likely to benefit from thrombolytic therapy and a high level of Lp-PLA2 indicates a subject likely to benefit from aggressive thrombolytic therapy, drug combinations or interventional and surgical approaches.

2. The method of claim 1 wherein the Lp-PLA2 mass level is determined.

3. The method of claim 1 wherein the Lp-PLA2 activity level is determined.

4. The method of claim 1 wherein a low level of Lp-PLA2 is less than or equal to 201.5 ng/ml and a high level of Lp-PLA2 is greater than 201.5 ng/mL.

5. (canceled)

6. The method of claim 1 wherein the subject has or is suspected of having coronary vascular disease (CVD).

7. The method of claim 6 wherein the CVD is selected from the group consisting of high blood pressure, coronary heart disease (CHD), myocardial infarction, stroke, transient ischemic attack (TIA), cerebrovascular accident (CVA), congenital cardiovascular defects and congestive heart failure.

8. (canceled)

9. The method of claim 1 wherein the subject is in an acute care setting.

10. (canceled)

11. The method of claim 1 wherein the thrombolytic therapy is selected for the subject within three to four hours of the subject having a myocardial infarction, stroke, TIA or CVA or a suspected myocardial infarction, stroke, TIA or CVA.

12. (canceled)

13. The method of claim 1 further comprising determining if the subject has a proximal vascular lesion or occlusion.

14. The method of claim 13 wherein the proximal vascular lesion or occlusion is determined by transcranial Doppler (TCD).

15. The method of claim 1 further comprising administering to a subject determined to have a low level of Lp-PLA2 thrombolytic therapy.

16. The method of claim 1 further comprising administering to a subject determined to have a high level of Lp-PLA2 aggressive thrombolytic therapy, drug combinations or interventional and surgical approaches.

17-18. (canceled)

19. A method of assessing functional outcome of a subject who has had or is suspected of having a myocardial infarction, stroke, TIA or CVA, said method comprising determining a level of Lp-PLA2 and presence of a proximal vascular lesion or occlusion in the subject, wherein the functional outcome of a subject with a high Lp-PLA2 level and a proximal vascular lesion or occlusion is functional dependence.

20. The method of claim 19 wherein the Lp-PLA2 mass level is determined.

21. The method of claim 19 wherein the Lp-PLA2 activity level is determined.

22. The method of claim 19 wherein a high level of Lp-PLA2 is greater than 201.5 ng/mL.

23-24. (canceled)

25. The method of claim 19 wherein the functional outcome of a subject is assessed in an acute care setting.

26-28. (canceled)

29. The method of claim 19 further comprising assessing neurological function in the subject.

30-37. (canceled)
